(19) United States
(12) Patent Application Publication
(10) Pub. No.: US 2016/0258010 A1
(43) Pub. Date: Sep. 8, 2016

(54) ALLELE SPECIFIC PCR ASSAY FOR DETECTION OF NUCLEOTIDE VARIANTS

(71) Applicants: Constantinos PANOUSIS, Pittsburgh, PA (US); Elias HALVAS, Pittsburgh, PA (US); John MELLORS, Pittsburgh, PA (US); University of Pittsburgh - Of the Commonwealth System of Higher Education, Pittsburgh, PA (US)

(72) Inventors: Constantinos Panousis, Pittsburgh, PA (US); Elias Halvas, Pittsburgh, PA (US); John Mellors, Pittsburgh, PA (US)

(73) Assignee: University of Pittsburgh - Of the Commonwealth System of Higher Education, Pittsburgh, PA (US)

(21) Appl. No.: 15/030,019

(22) PCT Filed: Oct. 20, 2014
(86) PCT No.: PCT/US14/61377

§ 371 Date: Apr. 15, 2016

Related U.S. Application Data
(60) Provisional application No. 61/892,993, filed on Oct. 18, 2013.

(51) Int. Cl. C12Q 1/68

(52) U.S. Cl. CPC C12Q 1/6858; C12Q 1/6851

(57) ABSTRACT

Described herein are improved methods for detecting one or more alleles of a target nucleic acid molecule in a biological sample. The methods can be used, for example, for detecting low-frequency drug resistance mutations of a target nucleic acid molecule in a biological sample from a subject receiving the drug. In several embodiments, the subject is a subject with an HIV-1 infection, and the method is a method of detecting one or more drug-resistance mutations in an HIV-1 reverse transcriptase gene. Oligonucleotide primers for use in the disclosed methods, and compositions comprising same, are also provided.

### CROSS REFERENCE TO RELATED APPLICATIONS

This application claims priority to U.S. Provisional Application No. 61/892,993, filed Oct. 18, 2013, which is incorporated by reference in its entirety.

### ACKNOWLEDGMENT OF GOVERNMENT SUPPORT

This invention was made with government support under Grant No. AI068633 awarded by the National Institutes of Health. The government has certain rights in the invention.

### FIELD

This application relates to the field of detecting minor or major genomic variants in a polymorphic background, particularly to methods and compositions for identifying drug-resistant viral strains.

### BACKGROUND

Variations in the nucleotide sequence of DNA impact if and how an organism develops diseases, and respond to pathogens, chemicals, drugs, vaccines and other agents.

Several assays have been developed for detection of the presence of low-frequency drug-resistant nucleotide mutations in a clinical sample, including Ultra-Deep Pyrosequencing (UDPS), Single Genome Sequencing (SGS), and Allele-Specific Polymerase Chain Reaction (ASPCR). UDPS is characterized by the sequencing of many genomes in a single run and the detection of low-frequency variants down to one percent. UDPS is more sensitive than standard sequencing and can lower the risk of virologic failure, but it is very expensive and requires extensive sequence alignment computation. SGS is characterized by the sequencing of 45-50 individual clones and a sensitivity of minor variant detection down to five percent. However, SGS sensitivity is limited by the number of clones tested, and the assay suffers from high cost and labor intensity. ASPCR is a quantitative real-time polymerase chain reaction (qPCR)-based assay where one of the amplification primers includes a 3′ nucleotide that is complementary to a mutant allele of interest and not complementary to the wild-type allele. Detection is achieved by the higher amplification efficiency on mutant sequence versus the wild type sequence. Although ASPCR assays are low cost and sensitive, the clinical utility of current ASPCR assays is limited due to false negative and positive artifacts caused by DNA polymorphisms that affect primer binding, as well as the inability to detect more than one polymorphism in a single assay.

### SUMMARY

Provided herein is a modified ASPCR assay that addresses many of the issues associated with current methodologies. The disclosed methods have increased sensitivity, minimize false positive results and high background, can detect linkage between two polymorphisms (e.g., drug resistance mutations) in a target nucleic acid molecule, and are designed to minimize variability associated with target polynucleotide polymorphism. In some embodiments, the methods can be used to detect a mutant allele of a target nucleic acid molecule that is associated with drug-resistance to a particular therapy, such as antiretroviral therapy for Human Immunodeficiency Virus (HIV)-1 infection.

Several embodiments include a method of detecting a mutant first allele of a target nucleic acid molecule in a biological sample. The method includes a test amplification and a control amplification. The test amplification includes amplifying the target nucleic acid molecule from the biological sample by qPCR using a test primer pair comprising a first plus primer comprising a locked nucleic acid at the 3′ end that is complementary to the mutant first allele of the target nucleic acid molecule, a mismatch nucleotide at the −1 position from the 3′ end that is not complementary to the target nucleic acid molecule, and remaining nucleotides that are complementary to the target nucleic acid molecule, and a reverse primer of the test primer pair. The control amplification includes amplifying the target nucleic acid molecule from the biological sample by qPCR using a control set of primers comprising the first primer pair, and a first control primer comprising a locked nucleic acid at the 3′ end that is complementary to a wildtype first allele corresponding to the mutant first allele, and remaining nucleotides that are the same as the first plus primer. The threshold cycle (Ct) value of the test amplification and the Ct value of the control amplification are measured. A difference between the Ct value of the test amplification and the Ct value of the control amplification is compared with a standard control generated using a mixture of target nucleic acid molecules comprising a pre-selected proportion of the mutant and wildtype first alleles to detect the first allele of the target nucleic acid molecule in the biological sample.

In several embodiments, detecting the mutant first allele of the target nucleic acid molecule in the biological sample comprises detecting a proportion of target nucleic acid molecules in the biological sample that comprises the first allele. In several embodiments, no more than 20% (such as no more than 1%) of the target nucleic acid molecules in the biological sample comprise the mutant first allele.

In other embodiments, the method can further include detecting a mutant second allele of the target nucleic acid molecule in the biological sample, wherein the reverse primer of the test primer pair is a second plus primer comprising a locked nucleic acid at the 3′ end that is complementary to the mutant second allele, a mismatch nucleotide at the −1 position from the 3′ end that is not complementary to the target nucleic acid molecule, and remaining nucleotides that are complementary to the target nucleic acid molecule; and the control set of primers further comprises a second control primer comprising a locked nucleic acid at the 3′ end that is complementary to a wildtype second allele corresponding to the mutant second allele, and remaining nucleotides that are the same as the second plus primer. The difference between the Ct values of the test and control amplifications are compared with a standard control generated using a mixture of target nucleic acid molecules comprising a pre-selected proportion of the mutant and wildtype first alleles and the mutant and wildtype second alleles to detect the mutant first and second alleles of the target nucleic acid molecule in the biological sample.

In a non-limiting example, the disclosed methods can be used to identify a K65R and/or M184V mutant allele of HIV-1 reverse transcriptase in a biological sample, for example, a biological sample from a subject with HIV-1 infection.

Reagents (for example, novel oligonucleotide primers and compositions comprising same) and kits for use in the disclosed methods are also provided.

The foregoing and other features and advantages of this disclosure will become more apparent from the following detailed description of several embodiments which proceeds with reference to the accompanying figures.

### BRIEF DESCRIPTION OF THE FIGURES

FIG. 1 is a graph illustrating discrimination between mutant (65R) and wild type (65K) templates for three ASPCR primer designs at two annealing temperatures of 55 and 61° C. The primers used included a locked nucleic acid at the 3′ end (LNA), a mismatch at the penultimate site (PEN), or a combination of the LNA and PEN (PLUS). Discriminatory power of the assay is expressed as ΔCt.

FIGS. 2A and 2B are a set of graphs illustrating validation of the disclosed ASPCR assay with M184V and M184I target nucleic acids. Both Plus and PEN primers for M184V and M184I were of identical sequence. PLUS primers were additionally modified with a 3′ end LNA. ASPCR was performed over a range of annealing temperatures ranging from 55° C. to 65° C. ΔCt corresponds to change in Ct.

FIGS. 3A-3C are a series of schematic diagrams illustrating the prior method for detecting total target DNA in an ASPCR assay, as well as the new method disclosed herein. (A) Old method; Assay is normalized against a total reaction either at a different or at the same site as the allele specific reaction. (B) Total primer missing the 3′ mismatch does not translate to same amplification efficiency, as mismatches present close to 3′ can have deleterious effects on amplification efficiency. (C) New method; Assay is normalized using the exact same amplicon and 3′ degenerative primers for the mutation in question.

FIG. 4 shows a set of graphs illustrating the reduction of background and limit of detection using a proof reading polymerase. Ten clinical samples were tested by the disclosed ASPCR assay for K65R and M184V using either a non-proof reading polymerase (Taq) or a proof-reading polymerase (Phusion).

FIGS. 5A and 5B are a graph and a schematic representation illustrating the detection of linked resistance mutations using the disclosed ASPCR assay. (A) Detection of linked K65R and M184V mutations by ASPCR utilizing a K65R allele specific forward primer and a M184V allele specific reverse primer. (B) Illustration of templates and normalization reaction for standards and samples.

FIG. 6 is a table showing a comparison between an older ASPCR method and the disclosed ASPCR assay for detection of mutant alleles in clinical samples. Percentage shown in parentheses is estimated based on electropherogram.

FIG. 7 is a table showing the validation of the disclosed ASPCR assay for detecting a M184V allele in pre-made viral mixtures of wild-type and M184 V HIV-1, spiked in human negative plasma.

FIG. 8 is a table showing the detection of linked K65R/M184V resistance mutations using the disclosed ASPCR assay.

FIG. 9 shows a schematic diagram and a set of graphs illustrating improved methods for normalization of ASPCR assays.

FIG. 10 is a set of graphs illustrating that the disclosed ASPCR assay can successfully detect mutant alleles from HIV-1 sub-types with polymorphic differences.

### SEQUENCE LISTING

The nucleic and amino acid sequences listed in the accompanying sequence listing are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. The Sequence Listing is submitted as an ASCII text file in the form of the file named “Sequence.txt” (˜20 kb), which was created on Oct. 17, 2014, which is incorporated by reference herein. In the accompanying sequence listing:

SEQ ID NOs: 1, 4, 6, 9, 11, 15-16, 19, and 39-40 are the nucleic acid sequences of allele-specific Plus primers including a mismatch nucleotide at the −1 position from the ′3 end and a locked nucleic acid at the 3′ end that is complementary to mutant allele sequence.

SEQ ID NOs: 2, 5, 7, 10, 12, 17, 20, and 41-42 are the nucleic acid sequences of wildtype-specific Control primers including a mismatch nucleotide at the −1 position from the ′3 end and a locked nucleic acid at the 3′ end that is complementary to wildtype sequence.

SEQ ID NOs: 11, 13, 15, 33-34, and 43-46 are the nucleic acid sequences of additional control primers including a mismatch nucleotide at the −1 position from the ′3 end and a locked nucleic acid at the 3′ end that is not complementary to wildtype or mutant allele sequence.

SEQ ID NOs: 3, 8, 14, 18, 37, and 38 are the nucleic acid sequences of reverse oligonucleotide primers.

SEQ ID NOs: 21-31, and 35 are the nucleic acid sequences of oligonucleotide primers.

SEQ ID NO: 32 is the nucleotide sequence of genomic DNA encoding wild-type HIV-1 reverse transcriptase (HXB2 strain).

SEQ ID NO: 36 is the amino acid sequence of wild-type HIV-1 reverse transcriptase p66 subunit (HXB2 strain).

### DETAILED DESCRIPTION

### I. Summary of Terms

Unless otherwise noted, technical terms are used according to conventional usage. Definitions of common terms in molecular biology may be found in Benjamin Lewin, Genes X, published by Jones & Bartlett Publishers, 2009; and Meyers et al. (eds.), The Encyclopedia of Cell Biology and Molecular Medicine, published by Wiley-VCH in 16 volumes, 2008; and other similar references.

As used herein, the singular forms “a,” “an,” and “the,” refer to both the singular as well as plural, unless the context clearly indicates otherwise. As used herein, the term “comprises” means “includes.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

3′ end: The end of a nucleic acid molecule that does not have a nucleotide bound to it 3′ of the terminal residue.

5′ end: The end of a nucleic acid sequence where the 5′ position of the terminal residue is not bound by a nucleotide.

Allele: A particular form of a genetic locus, distinguished from other forms by its particular nucleotide sequence, or one of the alternative polymorphisms found at a polymorphic site.

Allele-specific: A particular position of a nucleic acid sequence that, with reference oligonucleotides and primers, is complementary with an allele of a target polynucleotide sequence. Allele-specific primers are capable of discriminating between different alleles of a target polynucleotide. It is understood that several disclosed oligonucleotides include deliberate mismatches (at a different position than the allele-specific nucleotide) such that the oligonucleotide is not exactly complementary to the target polynucleotide. The function of the allele-specific oligonucleotides (e.g., plus primers) is to facilitate preferential hybridization and extension under PCR conditions of primers having the allele-specific nucleotide, or, alternatively, suppressing hybridization and extension of primers not having the allele-specific nucleotide.

Amplification: A technique that increases the number of copies of a nucleic acid molecule (such as an RNA or DNA). An example of amplification is polymerase chain reaction (PCR), in which a sample is contacted with a pair of oligonucleotide primers under conditions that allow for the hybridization of the primers to a nucleic acid template in the sample. The primers are extended under suitable conditions (e.g., in the presence of a polymerase enzyme and dNTPs), dissociated from the template, re-annealed, extended, and dissociated to amplify the number of copies of the nucleic acid. The product of amplification can be characterized by electrophoresis, restriction endonuclease cleavage patterns, oligonucleotide hybridization or ligation, and/or nucleic acid sequencing using standard techniques.

Other examples of amplification include quantitative real-time polymerase chain reaction (qPCR), strand displacement amplification, as disclosed in U.S. Pat. No. 5,744,311; transcription-free isothermal amplification, as disclosed in U.S. Pat. No. 6,033,881; repair chain reaction amplification, as disclosed in PCT publication WO 90/01069; ligase chain reaction amplification, as disclosed in European patent publication EP-A-320,308; gap filling ligase chain reaction amplification, as disclosed in U.S. Pat. No. 5,427,930; and NASBA RNA transcription-free amplification, as disclosed in U.S. Pat. No. 6,025,134. Several embodiments include multiplex qPCR assays, which are useful for amplifying and detecting multiple nucleic acid sequences in a single reaction.

Biological sample: A sample of biological material obtained from a subject. Biological samples include all clinical samples useful for detection of disease or infection (e.g., HIV infection) in subjects. Appropriate samples include any conventional biological samples, including clinical samples obtained from a human or veterinary subject. Exemplary samples include, without limitation, cells, cell lysates, blood smears, cytocentrifuge preparations, cytology smears, bodily fluids (e.g., blood, plasma, serum, saliva, sputum, urine, bronchoalveolar lavage, semen, cerebrospinal fluid (CSF), etc.), tissue biopsies or autopsies, fine-needle aspirates, and/or tissue sections. In a particular example, a biological sample is obtained from a subject having, suspected of having or at risk of having HIV infection.

Complementary. Complementary binding occurs when the base of one nucleic acid molecule forms a hydrogen bond to the base of another nucleic acid molecule. Normally, the base adenine (A) is complementary to thymidine (T) and uracil (U), while cytosine (C) is complementary to guanine (G). For example, the sequence 5′-ATCG-3′ of one ssDNA molecule can bond to 3′-TAGC-5′ of another ssDNA to form a dsDNA. In this example, the sequence 5′-ATCG-3′ is the reverse complement of 3′-TAGC-5′.

Nucleic acid molecules can be complementary to each other even without complete hydrogen-bonding of all bases of each molecule. For example, hybridization with a complementary nucleic acid sequence can occur under conditions of differing stringency in which a complement will bind at some but not all nucleotide positions. In particular examples disclosed herein, the complementary sequence is complementary at a labeled nucleotide, and at each nucleotide immediately flanking the labeled nucleotide.

Consists of or consists essentially of: With regard to a polynucleotide (such as a probe or primer), a polynucleotide consists essentially of a specified nucleotide sequence if it does not include any additional nucleotides. However, the polynucleotide can include additional non-nucleic acid components, such as labels (for example, fluorescent, radioactive, or solid particle labels), sugars or lipids. With regard to a polynucleotide, a polynucleotide that consists of a specified nucleotide sequence does not include any additional nucleotides, nor does it include additional non-nucleic acid components, such as lipids, sugars or labels.

Contacting: Placement in direct physical association, for example solid, liquid or gaseous forms. Contacting includes, for example, direct physical association of fully- and partially-solvated molecules.

Control: A sample or standard used for comparison with an experimental sample. In some embodiments, the control is a sample obtained from a healthy patient, or a sample from a subject with HIV. In some embodiments, the control is a sample including HIV nucleic acid. In other embodiments, the control is a biological sample obtained from a patient diagnosed with HIV. In still other embodiments, the control is a historical control or standard reference value or range of values (such as a previously tested control sample, such as a group of HIV patients with known prognosis or outcome, or group of samples that represent baseline or normal values, such as the presence or absence of HIV in a biological sample.

In some embodiments, the results of an ASPCR assay performed on a test sample using primer sets as described herein can be compared with a standard control (such as a standard curve) generated using an ASPCR assay with the same primer sets that is performed on a mixture of target nucleic acid molecules comprising a pre-selected proportion of mutant and wildtype alleles to detect the presense (or proportion) of a particular allele in the test sample.

Ct (threshold cycle): The PCR cycle number at which the fluorescence emission (dRn) exceeds a chosen threshold, which is typically 10 times the standard deviation of the baseline (this threshold level can, however, be changed if desired). The threshold cycle is when the system begins to detect the increase in the signal associated with an exponential growth of PCR product during the log-linear phase. This phase provides information about the reaction. The slope of the log-linear phase is a reflection of the amplification efficiency. The efficiency of the reaction can be calculated by the following equation: E=10^{(-1/slope)}, for example. The efficiency of the PCR should be 90-100% meaning doubling of the amplicon at each cycle. This corresponds to a slope of −3.1 to −3.6 in the Cₜ vs. log-template amount standard curve.

Detecting: To identify the existence, presence, or fact of something. General methods of detecting are known to the skilled artisan and may be supplemented with the protocols and reagents disclosed herein. For example, included herein are methods of detecting HIV in a biological sample, including detecting a particular HIV allele in a biological sample, such as a drug resistant allele. Detection can include a physical readout, such as fluorescence or a reaction output, or the results of PCR (such as qPCR) assay.

Detectable marker: A detectable molecule (also known as a label) that is conjugated directly or indirectly to a second molecule, such as a nucleic acid molecule, to facilitate detection of the second molecule. The person of ordinary skill in the art is familiar with detectable markers for labeling nucleic acid molecules and their use. For example, the detectable marker can be capable of detection by ELISA, spectrophotometry, flow cytometry, or microscopy. Specific, non-limiting examples of detectable markers include fluorophores, fluorescent proteins, chemiluminescent agents, enzymatic linkages, radioactive isotopes and heavy metals or compounds. In several embodiments, the detectable markers are designed for use with qPCR, such as multiplex qPCR. Various methods of labeling nucleic acid molecules are known in the art and may be used.

Diagnosis: The process of identifying a disease by its signs, symptoms and results of various tests. The conclusion reached through that process is also called “a diagnosis.” Forms of testing commonly performed include blood tests, medical imaging, urinalysis, and biopsy.

Human Immunodeficiency Virus (HIV): A retrovirus that causes immunosuppression in humans (HIV disease), and leads to a disease complex known as the acquired immunodeficiency syndrome (AIDS). “HIV disease” refers to a well-recognized constellation of signs and symptoms (including the development of opportunistic infections) in persons who are infected by an HIV virus, as determined by antibody or western blot studies. Laboratory findings associated with this disease include a progressive decline in T cells. HIV includes HIV type 1 (HIV-1) and HIV type 2 (HIV-2). Related viruses that are used as animal models include simian immunodeficiency virus (SW), and feline immunodeficiency virus (FIV). Treatment of HIV-1 with HAART has been effective in reducing the viral burden and ameliorating the effects of HIV-1 infection in infected individuals.

HXB2 numbering system: A reference numbering system for HIV protein and nucleic acid sequences, using the HIV-1 HXB2 strain sequences as a reference for all other HIV-1 strain sequences. The person of ordinary skill in the art is familiar with the HXB2 numbering system, and this system is set forth in “Numbering Positions in HIV Relative to HXB2CG,” Bette Korber et al., Human Retroviruses and AIDS 1998: A Compilation and Analysis of Nucleic Acid and Amino Acid Sequences. Korber B, Kuiken C L, Foley B, Hahn B, McCutchan F, Mellors J W, and Sodroski J, Eds. Theoretical Biology and Biophysics Group, Los Alamos National Laboratory, Los Alamos, N. Mex., which is incorporated by reference herein in its entirety. HXB2 is also known as: HXBc2, for HXB clone 2; HXB2R, in the Los Alamos HIV database, with the R for revised, as it was slightly revised relative to the original HXB2 sequence; and HXB2CG in GENBANK™, for HXB2 complete genome. The HIV-1 numbering used herein (e.g., the numbering for mutant alleles of HIV-1) is relative to the HXB2 numbering scheme.

Hybridization: The terms “annealing” and “hybridization” refer to the formation of base pairs between complementary regions of DNA, RNA, or between DNA and RNA of nucleic acids. Examples of annealing and hybridization include formation of base pairs between two separate nucleic acid molecules, as well as formation of base pairs between nucleic acids on a single nucleic acid molecule.

In some examples, hybridization is between two complementary nucleic acid sequences, for example nucleic acid sequences that are at least 90% complementary to each other, such as at least 91%, 92%, 93%, 94%, 95%, 96%, 97%, 98%, 99%, or 100% complementary to each other.

In additional embodiments, hybridization conditions resulting in particular degrees of stringency and specificity will vary depending upon the nature of the hybridization method and the composition and length of the hybridizing nucleic acid sequences. Generally, the temperature of hybridization and the ionic strength (such as the Na⁺ concentration) of the hybridization buffer will determine the stringency of hybridization. Calculations regarding hybridization conditions for attaining particular degrees of stringency are discussed in, e.g., Sambrook et al. (Molecular Cloning: A Laboratory Manual, 4^{th} ed, Cold Spring Harbor, N. Y., 2012) and Ausubel et al. (In Current Protocols in Molecular Biology, John Wiley & Sons, New York, through supplement 104, 2013). The following is an exemplary set of hybridization conditions and is not limiting:

Very High Stringency (Detects Sequences that Share at Least 90% Identity)
Hybridization: 5×SSC at 65° C. for 16 hours
Wash twice: 2×SSC at room temperature (RT) for 15 minutes each
Wash twice: 0.5×SSC at 65° C. for 20 minutes each
High Stringency (Detects Sequences that Share at Least 80% Identity)
Hybridization: 5×-6×SSC at 65° C.-70° C. for 16-20 hours
Wash twice: 2×SSC at RT for 5-20 minutes each
Wash twice: 1×SSC at 55° C.-70° C. for 30 minutes each
Low Stringency (Detects Sequences that Share at Least 50% Identity)
Hybridization: 6×SSC at RT to 55° C. for 16-20 hours
Wash at least twice: 2×-3×SSC at RT to 55° C. for 20-30 minutes each.

In some embodiments, the probes and primers disclosed herein can hybridize to nucleic acid molecules under low stringency, high stringency, and very high stringency conditions.

Inhibiting or treating a disease: Inhibiting the full development of a disease or condition, for example, in a subject who is at risk for a disease such as HIV. “Treatment” refers to a therapeutic intervention that ameliorates a sign or symptom of a disease or pathological condition after it has begun to develop. The term “ameliorating,” with reference to a disease or pathological condition, refers to any observable beneficial effect of the treatment. The beneficial effect can be evidenced, for example, by a delayed onset of clinical symptoms of the disease in a susceptible subject, a reduction in severity of some or all clinical symptoms of the disease, a slower progression of the disease, a reduction in viral titer, an improvement in the overall health or well-being of the subject, or by other parameters well known in the art that are specific to the particular disease. A “prophylactic” treatment is a treatment administered to a subject who does not exhibit signs of a disease or exhibits only early signs for the purpose of decreasing the risk of developing pathology.

Isolated: An “isolated” biological component (such as a nucleic acid molecule or protein) has been substantially separated or purified away from other biological components in the cell of the organism in which the component naturally occurs. The term “isolated” does not require absolute purity. Nucleic acids and proteins that have been “isolated” include nucleic acids and proteins purified by standard purification methods. The term also embraces nucleic acids and proteins prepared by recombinant expression in a host cell, as well as chemically synthesized nucleic acids.

Locked nucleic acid (LNA): A synthetic nucleic acid analogue, incorporating “internally bridged” nucleoside analogues, for example, 2′-4′- and 3′-4′-linked and other bicyclic sugar modifications. LNA exhibits greater thermal stability when paired with DNA, than do conventional DNA/DNA heteroduplexes and can be synthesized on conventional nucleic acid synthesizing machines. LNA molecules, and methods of synthesizing and using LNA molecules in oligonucleotide, are known in the art and are disclosed, for example, in the following publications: U.S. Pat. Nos. 6,316,198; 6,794,499; 7,034,133; 7,060,809; and 7,034,133; WO 98/22489; WO 98/39352; WO 99/14226; Nielsen et al., J. Chem. Soc. Perkin Trans. 1, 3423 (1997); Koshkin et al., Tetrahedron Letters 39, 4381 (1998); Singh & Wengel, Chem. Commun, 1247 (1998); and Singh et al., Chem. Commun. 455 (1998); the contents of which are incorporated herein by reference in their entirety.

Mismatch nucleotide: a nucleotide that is not complementary to the corresponding nucleotide of the opposite polynucleotide strand.

Multiplex qPCR: Amplification and detection of multiple nucleic acid species in a single qPCR reaction. By multiplexing, multiple target nucleic acids can be amplified in single tube. In some examples, multiplex PCR permits the simultaneous detection of the multiple HIV alleles, such as a drug-resistant allele and a non-drug resistant allele, or multiple drug resistant alleles.

Mutation: Any change of a nucleic acid sequence as a source of genetic variation. For example, mutations can occur within a gene or chromosome, including specific changes in non-coding regions of a chromosome, for instance changes in or near regulatory regions of genes. Types of mutations include, but are not limited to, base substitution point mutations (which are either transitions or transversions), deletions, and insertions. Missense mutations are those that introduce a different amino acid into the sequence of the encoded protein; nonsense mutations are those that introduce a new stop codon; and silent mutations are those that introduce the same amino acid often with a base change in the third position of the codon. In the case of insertions or deletions, mutations can be in-frame (not changing the frame of the overall sequence) or frame shift mutations, which may result in the misreading of a large number of codons (and often leads to abnormal termination of the encoded product due to the presence of a stop codon in the alternative frame).

Nucleic acid: A deoxyribonucleotide or ribonucleotide polymer, which can include analogues of natural nucleotides that hybridize to nucleic acid molecules in a manner similar to naturally occurring nucleotides. In a particular example, a nucleic acid molecule is a single stranded (ss) DNA or RNA molecule, such as a probe or primer. In another particular example, a nucleic acid molecule is a double stranded (ds) nucleic acid, such as a target nucleic acid. Examples of modified nucleic acids are those with altered sugar moieties, such as a locked nucleic acid (LNA).

Nucleotide: The fundamental unit of nucleic acid molecules. A nucleotide includes a nitrogen-containing base attached to a pentose monosaccharide with one, two, or three phosphate groups attached by ester linkages to the saccharide moiety.

The major nucleotides of DNA are deoxyadenosine 5′-triphosphate (dATP or A), deoxyguanosine 5′-triphosphate (dGTP or G), deoxycytidine 5′-triphosphate (dCTP or C) and deoxythymidine 5′-triphosphate (dTTP or T). The major nucleotides of RNA are adenosine 5′-triphosphate (ATP or A), guanosine 5′-triphosphate (GTP or G), cytidine 5′-triphosphate (CTP or C) and uridine 5′-triphosphate (UTP or U).

Nucleotides include those nucleotides containing modified bases, modified sugar moieties and modified phosphate backbones, as known in the art.

Examples of modified base moieties which can be used to modify nucleotides at any position on its structure include, but are not limited to: 5-fluorouracil, 5-bromouracil, 5-chlorouracil, 5-iodouracil, hypoxanthine, xanthine, acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N˜6-sopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, methoxyarninomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid, pseudouracil, queosine, 2-thiocytosine, 5-methyl-2-thiouracil, 2-thiouracil, 4-thiouracil, 5-methyluracil, uracil-5-oxyacetic acid methylester, uracil-S-oxyacetic acid, 5-methyl-2-thiouracil, 3-(3-amino-3-N-2-carboxypropyl) uracil, and 2,6-diaminopurine.

Examples of modified sugar moieties which may be used to modify nucleotides at any position on its structure include, but are not limited to: arabinose, 2-fluoroarabinose, xylose, and hexose, or a modified component of the phosphate backbone, such as phosphorothioate, a phosphorodithioate, a phosphoramidothioate, a phosphoramidate, a phosphordiamidate, a methylphosphonate, an alkyl phosphotriester, or a formacetal or analog thereof.

Conventional notation is used herein to describe nucleotide sequences: the left-hand end of a single-stranded nucleotide sequence is the 5′-end; the left-hand direction of a double-stranded nucleotide sequence is referred to as the 5′-direction. The direction of 5′ to 3′ addition of nucleotides to nascent RNA transcripts is referred to as the transcription direction. The DNA strand having the same sequence as an mRNA is referred to as the “coding strand;” sequences on the DNA strand having the same sequence as an mRNA transcribed from that DNA and which are located 5′ to the 5′-end of the RNA transcript are referred to as “upstream sequences;” sequences on the DNA strand having the same sequence as the RNA and which are 3′ to the 3′ end of the coding RNA transcript are referred to as “downstream sequences.” Unless denoted otherwise, whenever a polynucleotide sequence is represented, it will be understood that the nucleotides are in 5′ to 3′ orientation from left to right.

Oligonucleotide probes and primers: A probe includes an isolated nucleic acid (usually of 100 or fewer nucleotide residues) attached to a detectable label or reporter molecule, which is used to detect a complementary target nucleic acid molecule by hybridization and detection of the label or reporter. Isolated oligonucleotide probes (which as defined herein also include the complementary sequence and corresponding RNA sequences) are of use for detection of HIV sequences. Typically, probes are at least about 10 nucleotides in length, such as at least about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or about 100 nucleotides in length. For example, a probe can be about 10-100 nucleotides in length, such as, 12-15, 12-20, 12-25, 12-30, 12-35, 12-40, 12-45, 12-50, 12-80, 14-15, 14-16, 14-18, 14-20, 14-25, 14-30, 15-16, 15-18, 15-20, 15-25, 15-30, 15-35, 15-40, 15-45, 15-50, 15-80, 16-17, 16-18, 16-20, 16-22, 16-25, 16-30, 16-40, 16-50, 17-18, 17-20, 17-22, 17-25, 17-30, 18-19, 18-20, 18-22, 18-25, 18-30, 19-20, 19-22, 19-25, 19-30, 20-21, 20-22, 20-25, 20-30, 20-35, 20-40, 20-45, 20-50, 20-80, 21-22, 21-25, 21-30, 22-25, 22-30, 22-40, 22-50, 23-24, 23-25, 23-30, 24-25, 24-30, 25-35, 25-30, 25-35, 25-40, 25-45, 25-50 or 25-80 nucleotides in length.

Primers are nucleic acid molecules, usually DNA oligonucleotides of about 10-50 nucleotides in length (longer lengths are also possible). Typically, primers are at least about 10 nucleotides in length, such as at least about 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or about 50 nucleotides in length. For example, a primer can be about 10-50 nucleotides in length, such as, 12-15, 12-20, 12-25, 12-30, 12-35, 12-40, 12-45, 12-50, 14-15, 14-16, 14-18, 14-20, 14-25, 14-30, 15-16, 15-18, 15-20, 15-25, 15-30, 15-35, 15-40, 15-45, 15-50, 16-17, 16-18, 16-20, 16-22, 16-25, 16-30, 16-40, 16-50, 17-18, 17-20, 17-22, 17-25, 17-30, 18-19, 18-20, 18-22, 18-25, 18-30, 19-20, 19-22, 19-25, 19-30, 20-21, 20-22, 20-25, 20-30, 20-35, 20-40, 20-45, 20-50, 21-22, 21-25, 21-30, 22-25, 22-30, 22-40, 22-50, 23-24, 23-25, 23-30, 24-25, 24-30, 25-30, 25-35, 25-40 or 25-45, 25-50 nucleotides in length.

Probes and primers can also be of a maximum length, for example no more than 15, 25, 25, 40, 50, 75 or 100 nucleotides in length.

Primers may be annealed to a complementary target DNA strand by nucleic acid hybridization to form a hybrid between the primer and the target DNA strand, and then extended along the target DNA strand by a DNA polymerase enzyme. One of skill in the art will appreciate that the hybridization specificity of a particular probe or primer typically increases with its length. Thus, for example, a probe or primer including 20 consecutive nucleotides typically will anneal to a target with a higher specificity than a corresponding probe or primer of only 15 nucleotides. In some embodiments, probes and primers are used in combination in a qPCR reaction.

Plus Primer: An oligonucleotide primer for use in allele-specific PCR. A plus primer includes a locked nucleic acid at the 3′ position that is complementary to a mutant allele of a target nucleic acid, and a mismatch nucleotide at the −1 position from the 3′ end that is not complementary to the target nucleic acid molecule.

Primer pair: Two primers (one “forward” and one “reverse”) that can be used for amplification of a nucleic acid sequence, for example by polymerase chain reaction (PCR) or other in vitro nucleic-acid amplification methods. The forward and reverse primers of a primer pair do not hybridize to overlapping complementary sequences on the target nucleic acid sequence.

Polymorphism: A variation in a gene sequence. The polymorphisms can be those variations (DNA sequence differences, e.g., substitutions, deletions, or insertions) which are generally found between individuals or different ethnic groups and geographic locations which, while having a different sequence, produce functionally equivalent gene products. The term can also refer to variants in the sequence which can lead to gene products that are not functionally equivalent and/or have altered function. Polymorphisms also encompass variations which can be classified as alleles and/or mutations which either produce no gene product or an inactive gene product or an active gene product produced at an abnormal rate or in an inappropriate tissue or in response to an inappropriate stimulus. Alleles are the alternate forms that occur at the polymorphism. In one non-limiting example, a polymorphism is a mutation that confers resistance to a particular drug, for example, a HIV-1 therapeutic.

Polymorphisms can be referred to, for instance, by the nucleotide position at which the variation exists, by the change in amino acid sequence caused by the nucleotide variation, or by a change in some other characteristic of the nucleic acid molecule or protein that is linked to the variation.

Proof-Reading Polymerase: A polymerase enzyme with 3′ to 5′ exonuclease activity. A Non-limiting example of a proof-reading polymerase include Taq polymerase. Proof-reading polymerases with 3′ to 5′ exonuclease activity are known to the person of ordinary skill in the art and are commercially available, for example, from New England Biolabs, Ipswich, Mass.

Real-Time PCR (qPCR): A method for detecting and measuring products generated during each cycle of a PCR, which are proportionate to the amount of template nucleic acid prior to the start of PCR. The information obtained, such as an amplification curve, can be used to determine the presence of a target nucleic acid (such as a HIV nucleic acid or polymorphism thereof) and/or quantitate the initial amounts of a target nucleic acid sequence. Exemplary procedures for qPCR can be found in “Quantitation of DNA/RNA Using Real-Time PCR Detection” published by Perkin Elmer Applied Biosystems (1999); PCR Protocols (Academic Press, New York, 1989); A-Z of Quantitative PCR, Bustin (ed.), International University Line, La Jolla, Calif., 2004; and Quantitative Real-Time PCR in Applied Microbiology, Filion (Ed), Caister Academic Press, 2012.

In some examples, the amount of amplified target nucleic acid (for example a HIV nucleic acid) is detected using a labeled probe, such as a probe labeled with a fluorophore, for example a TAQMAN® probe. In other examples, the amount of amplified target nucleic acid (for example a HIV nucleic acid) is detected using a DNA intercalating dye. The increase in fluorescence emission is measured in real-time, during the course of the qPCR. This increase in fluorescence emission is directly related to the increase in target nucleic acid amplification. In some examples, the change in fluorescence (Delta Rn; dRn; ΔRn) is calculated using the equation dRn=Rn⁺−Rn⁻, with Rn⁺ being the fluorescence emission of the product at each time point and Rn⁻ being the fluorescence emission of the baseline. The dRn values are plotted against cycle number, resulting in amplification plots for each sample. The threshold cycle (Ct) is the PCR cycle number at which the fluorescence emission (dRn) exceeds a chosen threshold, which is typically 10 times the standard deviation of the baseline (this threshold level can, however, be changed if desired).

The threshold cycle is when the system begins to detect the increase in the signal associated with an exponential growth of PCR product during the log-linear phase. This phase provides information about the reaction. The slope of the log-linear phase is a reflection of the amplification efficiency. The efficiency of the reaction can be calculated by the following equation: E=10^{(-1/slope)} for example. The efficiency of the PCR should be 90-100% meaning doubling of the amplicon at each cycle. This corresponds to a slope of −3.1 to −3.6 in the Cₜ vs. log-template amount standard curve.

Recombinant: A recombinant nucleic acid is one that has a sequence that is not naturally occurring or has a sequence that is made by an artificial combination of two otherwise separated segments of sequence. This artificial combination can be accomplished by chemical synthesis or, more commonly, by the artificial manipulation of isolated segments of nucleic acids, e.g., by genetic engineering techniques.

Sensitivity and specificity: Statistical measurements of the performance of a binary classification test. Sensitivity measures the proportion of actual positives which are correctly identified (e.g., the percentage of samples that are identified as including nucleic acid from a particular virus). Specificity measures the proportion of negatives which are correctly identified (e.g., the percentage of samples that are identified as not including nucleic acid from a particular virus).

Sequence identity: The similarity between two nucleic acid sequences is expressed in terms of the similarity between the sequences, otherwise referred to as sequence identity. Sequence identity is frequently measured in terms of percentage identity, similarity, or homology; a higher percentage identity indicates a higher degree of sequence similarity.

The NCBI Basic Local Alignment Search Tool (BLAST), Altschul et al., J. Mol. Biol. 215:403-10, 1990, is available from several sources, including the National Center for Biotechnology Information (NCBI, Bethesda, Md.), for use in connection with the sequence analysis programs blastp, blastn, blastx, tblastn and tblastx. It can be accessed through the NCBI website. A description of how to determine sequence identity using this program is also available on the website.

When less than the entire sequence is being compared for sequence identity, homologs will typically possess at least 75% sequence identity over short windows of 10-20 amino acids, and can possess sequence identities of at least 85% or at least 90% or 95% depending on their similarity to the reference sequence. Methods for determining sequence identity over such short windows are described on the NCBI website.

These sequence identity ranges are provided for guidance only; it is entirely possible that strongly significant homologs could be obtained that fall outside of the ranges provided.

An alternative indication that two nucleic acid molecules are closely related is that the two molecules hybridize to each other under stringent conditions. Stringent conditions are sequence-dependent and are different under different environmental parameters. Generally, stringent conditions are selected to be about 5° C. to 20° C. lower than the thermal melting point (Tm) for the specific sequence at a defined ionic strength and pH. The Tm is the temperature (under defined ionic strength and pH) at which 50% of the target sequence hybridizes to a perfectly matched probe. Conditions for nucleic acid hybridization and calculation of stringencies can be found in Sambrook et al.; and Tijssen, Hybridization With Nucleic Acid Probes, Part I: Theory and Nucleic Acid Preparation, Laboratory Techniques in Biochemistry and Molecular Biology, Elsevier Science Ltd., 1993.

Signal: A detectable change or impulse in a physical property that provides information. In the context of the disclosed methods, examples include electromagnetic signals such as light, for example light of a particular quantity or wavelength. In certain examples, the signal is the disappearance of a physical event, such as quenching of light.

Single nucleotide polymorphism (SNP): The polynucleotide sequence variation present at a single nucleotide residue within different alleles of the same genomic sequence. This variation may occur within the coding region or non-coding region (i.e., in the promoter region) or an intergenic (between genes) sequence of a genomic sequence. Detection of one or more SNP allows differentiation of different alleles of a single genomic sequence. Most common SNPs have only two alleles.

SNPs within a coding sequence will not necessarily change the amino acid sequence of the protein that is produced, due to degeneracy of the genetic code. A SNP in which both forms lead to the same polypeptide sequence is termed “synonymous” (sometimes called a silent mutation)—if a different polypeptide sequence is produced they are “nonsynonymous”. A nonsynonymous change may either be missense or “nonsense”, where a missense change results in a different amino acid, while a nonsense change results in a premature stop codon.

Subject: Any mammal, such as humans, non-human primates, pigs, sheep, cows, rodents and the like. In two non-limiting examples, a subject is a human subject or a murine subject. Thus, the term “subject” includes both human and veterinary subjects. A immunocompromised subject is a subject with a suppressed immune system, such as a subject with HIV.

Target nucleic acid molecule: A nucleic acid molecule whose detection, quantitation, qualitative detection, or a combination thereof, is intended. The nucleic acid molecule need not be in a purified form. Various other nucleic acid molecules can also be present with the target nucleic acid molecule. For example, the target nucleic acid molecule can be a specific nucleic acid molecule (which can include RNA or DNA), the amplification of which is intended. In some examples, a target nucleic acid includes a region of the HIV genome that includes an allele specific mutation that results in drug resistance. Purification or isolation of the target nucleic acid molecule, if needed, can be conducted by methods known to those in the art, such as by using a commercially available purification kit or the like.

Under conditions sufficient for: A phrase that is used to describe any environment that permits a desired activity. In one example the desired activity is amplification of a nucleic acid molecule.

Wild-type: The genotype or phenotype that is most prevalent in nature. The naturally occurring, non-mutated version of a nucleic acid sequence. Among multiple alleles, the allele with the greatest frequency within the population is usually the wild-type. The term “native” can be used as a synonym for “wild-type.”

### II. Detecting Alleles of a Nucleic Acid Molecule

A method is disclosed for detecting alleles of a target nucleic acid molecule using a novel allele-specific PCR (ASPCR) assay. The methods are useful, for example, for identifying and diagnosing a subject with a particular polymorphism, such as a drug resistant allele of HIV-1.

The principle of ASPCR is based on the differences of amplification efficiencies using mutation-specific and wt-specific oligonucleotide primers in a qPCR assay with a common target nucleic acid template from a biological sample. The resulting “test” amplification is compared with a “control” amplification that detects total amount of the target template in the biological sample to normalize the amplification assay. The difference between the cycle number when the test amplification reaches a threshold amount of amplified DNA, and the cycle number when the control amplification reached the threshold amount of amplified DNA is the ΔCt value for the ASPCR assay. The ΔCt value for the ASPCR assay can be compared to a standard control (or curve) generated using a mixture of target nucleic acid molecules comprising a pre-selected proportion of the mutant and wildtype first alleles to detect the mutant first allele of the target nucleic acid molecule in the biological sample.

The disclosed ASPCR assay includes use of one or more “Plus” primers, which include a penultimate mismatch at the −1 position from the 3′ end, and a locked nucleic acid (LNA) at the 3′ end that complements with an allele of interest in the target nucleic acid. As illustrated in Example 1, use of the Plus primers increases the difference in amplification efficiency between Mutant and Wild Type by 3-5 cycles as calculated by qPCR; thereby increasing selectivity and sensitivity of amplification for the allele of interest.

The disclosed ASPCR assay utilizes improved methods of normalizing the amount of target nucleic acid molecules in a biological sample to provide for increased sensitivity of the assay. In several embodiments the method includes a control qPCR assay utilizing a mixture of primers for at least one half of the primer pair (the forward primer set) used for the ASPCR assay, as follows:

A) two primers: a plus primer (including a 3′ LNA specific for the allele of interest and a mismatch at the −1 position) and a corresponding control primer (including a 3′ LNA specific for the wild-type sequence and the same mismatch at the −1 position). The plus and the control primers are identical except for the ′3 end position (which is specific for the allele of interest in the Plus primer and specific for wild-type sequence in the control primer); or

B) four primers: a plus primer (including a 3′ LNA specific for the allele of interest and a mismatch at the −1 position), and three additional primers, including the remaining other nucleotides as LNAs at the 3′ end, and the same mismatch at the −1 position. The four primers are identical except for the ′3 end position.

The remaining (reverse) half of the primer pair for the normalization qPCR assay includes either a single primer having a sequence common to both wild-type and mutant allelic DNA, or in the case of a ASPCR assay that can detect two alleles, the remaining half for the normalization qPCR assay can include two or four primers as described above that are complementary to the second allele targeted by the linked ASPCR assay. Using these primer mixtures, a standard curve can be generated by measuring the Delta Ct between the ASPCR and the Total-PCR of a series of standard templates, for example ranging from 0.01% mixtures of mutant/wild-type up to 100% mutant. As illustrated in Example 1, the novel control amplification provides for improved sensitivity and specificity compared to prior control amplification reactions.

In some embodiments, the disclosed methods can predict with a sensitivity of at least 90% and/or a specificity of at least 90% for the identity of a mutant allele of a nucleic acid molecule in a biological sample, such as a sensitivity of at least 91%, 92%, 93%, 94%, 95%, 96%, 97%, 98%, 99%, or even 100% and a specificity of at least of at least 91%, 292%, 93%, 94%, 95%, 96%, 97%, 98%, 99%, or even 100%.

In further embodiments, the disclosed methods can detect the proportion (such as no more than 20%, for example, no more than 15%, no more than 10%, no more than 5%, no more than 4%, no more than 3%, no more than 2%, no more than 1%, no more than 0.1%, or less) of target nucleic acid molecules in a biological sample including a mutant allele of interest.

In several embodiments, a method of detecting a first allele of a target nucleic acid molecule in a biological sample is provided. The method includes a test amplification and a control amplification. The test amplification includes amplifying the target nucleic acid molecule from the biological sample by qPCR using a test primer pair comprising (1) a first plus primer comprising a locked nucleic acid at the 3′ end that is complementary to the first allele, a mismatch nucleotide at the −1 position from the 3′ end, and remaining nucleotides complementary to the target nucleic acid molecule, and (2) a reverse primer of the test primer pair. The control amplification includes amplifying the target nucleic acid molecule from the biological sample by qPCR using a control set of primers comprising (1) the first primer pair, and (2) a first control primer comprising a locked nucleic acid at the 3′ end that is complementary to the first allele, and remaining nucleotides the same as the first plus primer. The Ct value of the test amplification and the Ct value of the control amplification are measured, and the difference between the Ct values (ΔCt) is determined. The ΔCt value for the ASPCR assay can be compared to a standard curve generated using known mixtures of mutant and wild-type target nucleic acid molecules to determine the proportion of the mutant and/or wild-type allele of the target nucleic acid molecule in the biological sample.

In additional embodiments, the control set of primers can further include additional control primers. For example, the control amplification can include a third control primer, comprising a locked nucleic acid at the 3′ end that is not complementary to the mutant or wildtype first allele, and remaining nucleotides that are the same as the first plus primer; and a fourth control primer, comprising a locked nucleic acid at the 3′ end that is not complementary to the mutant or wildtype first allele and is not the same as the locked nucleic acid of the third control primer, and remaining nucleotides that are the same as the first plus primer. The additional control primers provide increase sensitivity to the ASPCR assay.

In other embodiments, the assay can be used to detect a second allele of the target nucleic acid molecule in the biological sample. For example, the ASPCR can be used to detect two linked drug-resistance alleles of the target nucleic acid molecule. In some such embodiments, the reverse primer of the test primer pair can be a second plus primer comprising a locked nucleic acid at the 3′ end that is complementary to the mutant second allele, a mismatch nucleotide at the −1 position from the 3′ end that is not complementary to the target nucleic acid molecule, and remaining nucleotides that are complementary to the target nucleic acid molecule. Further, the control set of primers includes a second control primer comprising a locked nucleic acid at the 3′ end that is complementary to a wildtype second allele corresponding to the mutant second allele, and remaining nucleotides that are the same as the second plus primer. In such assays, the difference between the Ct values of the test and control amplifications are compared with a standard control generated using a mixture of target nucleic acid molecules comprising a pre-selected proportion of the mutant and wildtype first alleles and the mutant and wildtype second alleles to detect the mutant first and second alleles of the target nucleic acid molecule in the biological sample

In additional embodiments including detection of two alleles of the target nucleic acid molecule, the control set of primers can further include additional control primers. For example, the control set of primers can include a third control primer, comprising a locked nucleic acid at the 3′ end that is not complementary to the mutant or wildtype first allele, and remaining nucleotides that are the same as the first plus primer, as well as a fourth control primer, comprising a locked nucleic acid at the 3′ end that is not complementary to the mutant or wildtype first allele and is not the same as the locked nucleic acid of the third control primer, and remaining nucleotides that are the same as the first plus primer. Optionally, the control set of primers can further include a fifth control primer, comprising a locked nucleic acid at the 3′ end that is not complementary to the mutant or wildtype second allele, and remaining nucleotides that are the same as the second plus primer, as well as a sixth control primer, comprising a locked nucleic acid at the 3′ end that is not complementary to the mutant or wildtype second allele and is not the same as the locked nucleic acid of the fifth control primer, and remaining nucleotides that are the same as the second plus primer. The additional control primers provide increase sensitivity to the ASPCR assay.

In some embodiments, a non-proof-reading polymerase (e.g., Taq polymerase) can be used in the test and/or control amplification steps of the disclosed methods.

In additional embodiments, the assay can include a first round amplification of template DNA from the biological sample prior to the test or control amplifications, wherein the first round amplification comprises use of a proof-reading DNA polymerase (e.g., Phusion polymerase, sold by New England Biolabs). The product of the first round amplification is then used as template for the test and control amplifications as described herein.

### A. Detecting HIV Alleles

Even though treatment of HIV infections with antiretroviral (ARV) drugs has improved the clinical outcomes of HIV-1 infected patients considerably, their efficacy is still limited, due to the development and transmission of HIV-1 drug-resistant variants. High replication rates (i.e. production of >109 virions per day) coupled to the error-prone nature of HIV-1 Reverse Transcriptase (RT), frequently leads to the production of randomly generated mutations and a population of HIV-1 variants within a patient commonly referred to as the “quasispecies.” Furthermore, some of these randomly generated mutations can confer high level drug resistance to certain drugs (i.e. lamivudine, efavirenz, or nevirapine) by means of a single nucleotide substitution within the viral genome. These HIV resistance variants are believed to preexist even prior to the initiation of drug therapy and are usually the first to appear during virologic failure under treatment. In contrast, resistance to other antiretroviral drugs such as protease inhibitors and/or combination therapies, requires the accumulation of multiple mutations on the HIV genome.

Accordingly, in several embodiments the disclosed methods are utilized for detection of one or more alleles of HIV, such as a mutant allele of HIV-1 or HIV-2 that confers drug resistance. In some embodiments, the methods are used to detect one or more mutant alleles of HIV-1 reverse transcriptase. An exemplary nucleic acid sequence encoding a wild-type HIV-1 reverse transcriptase (HXB2 strain) is set forth as SEQ ID NO: 32:

cccattagccctattgagactgtaccagtaaaattaaagccaggaatgga
tggcccaaaagttaaacaatggccattgacagaagaaaaaataaaagcat
tagtagaaatttgtacagagatggaaaaggaagggaaaatttcaaaaatt
gggcctgaaaatccatacaatactccagtatttgccataaagaaaaaaga
cagtactaaatggagaaaattagtagatttcagagaacttaataagagaa
ctcaagacttctgggaagttcaattaggaataccacatcccgcagggtta
aaaaagaaaaaatcagtaacagtactggatgtgggtgatgcatatttttc
agttcccttagatgaagacttcaggaagtatactgcatttaccataccta
gtataaacaatgagacaccagggattagatatcagtacaatgtgcttcca
cagggatggaaaggatcaccagcaatattccaaagtagcatgacaaaaat
cttagagccttttagaaaacaaaatccagacatagttatctatcaataca
tggatgatttgtatgtaggatctgacttagaaatagggcagcatagaaca
aaaatagaggagctgagacaacatctgttgaggtggggacttaccacacc
agacaaaaaacatcagaaagaacctccattcctttggatgggttatgaac
tccatcctgataaatggacagtacagcctatagtgctgccagaaaaagac
agctggactgtcaatgacatacagaagttagtggggaaattgaattgggc
aagtcagatttacccagggattaaagtaaggcaattatgtaaactcctta
gaggaaccaaagcactaacagaagtaataccactaacagaagaagcagag
ctagaactggcagaaaacagagagattctaaaagaaccagtacatggagt
gtattatgacccatcaaaagacttaatagcagaaatacagaagcaggggc
aaggccaatggacatatcaaatttatcaagagccatttaaaaatctgaaa
acaggaaaatatgcaagaatgaggggtgcccacactaatgatgtaaaaca
attaacagaggcagtgcaaaaaataaccacagaaagcatagtaatatggg
gaaagactcctaaatttaaactgcccatacaaaaggaaacatgggaaaca
tggtggacagagtattggcaagccacctggattcctgagtgggagtttgt
taatacccctcccttagtgaaattatggtaccagttagagaaagaaccca
tagtaggagcagaaaccttctatgtagatggggcagctaacagggagact
aaattaggaaaagcaggatatgttactaatagaggaagacaaaaagttgt
caccctaactgacacaacaaatcagaagactgagttacaagcaatttatc
tagctttgcaggattcgggattagaagtaaacatagtaacagactcacaa
tatgcattaggaatcattcaagcacaaccagatcaaagtgaatcagagtt
agtcaatcaaataatagagcagttaataaaaaaggaaaaggtctatctgg
catgggtaccagcacacaaaggaattggaggaaatgaacaagtagataaa
ttagtcagtgctggaatcaggaaagtacta

An exemplary amino acid sequence of a wild-type HIV-1 reverse transcriptase (HXB2 strain, p66 subunit) is set forth as SEQ ID NO: 36:

PISPIETVPVKLKPGMDGPKVKQWPLTEEKIKALVEICTEMEKEGKISKI
GPENPYNTPVFAIKKKDSTKWRKLVDFRELNKRTQDFWEVQLGIPHPAGL
KKKKSVTVLDVGDAYFSVPLDEDFRKYTAFTIPSINNETPGIRYQYNVLP
QGWKGSPAIFQSSMTKILEPFRKQNPDIVIYQYMDDLYVGSDLEIGQHRT
KIEELRQHLLRWGLTTPDKKHQKEPPFLWMGYELHPDKWTVQPIVLPEKD
SWTVNDIQKLVGKLNWASQIYPGIKVRQLCKLLRGTKALTEVIPLTEEAE
LELAENREILKEPVHGVYYDPSKDLIAEIQKQGQGQWTYQIYQEPFKNLK
TGKYARMRGAHTNDVKQLTEAVQKITTESIVIWGKTPKFKLPIQKETWET
WWTEYWQATWIPEWEFVNTPPLVKLWYQLEKEPIVGAETFYVDGAANRET
KLGKAGYVTNRGRQKVVTLTDTTNQKTELQAIYLALQDSGLEVNIVTDSQ
YALGIIQAQPDQSESELVNQIIEQLIKKEKVYLAWVPAHKGIGGNEQVDK
LVSAGIRKVL

Several drug resistant HIV-1 mutations are known to the person of ordinary skill in the art, see, e.g., Johnson et al., “Update of the drug resistance mutations in HIV-1: March 2013”, Top Antivir Med. 21(1):6-14, 2013, which is incorporated by reference herein in its entirety. Further updates to the list of drug resistant HIV-1 mutations can be found at www.iasusa.org. Exemplary drug resistant HIV-1 mutations include, but are not limited to, the following mutant alleles of HIV-1 reverse transcriptase: K65R, M184V, M184I, M41L, A62V, K65N, K65E, D67N, D67G, D67E, T69I, T69D, K70R, K70E, K70G, K70T, K70N, K70Q, L74V, L74I, V75I, V75M, V75T, F77L, L100I, K101P, K103N, K103S, V106M, Y115F, F116Y, Q151M, Y181C, Y181I, Y188L, G190S, G190A, L210W, T215Y, T215F, T215E, T215I, T215C, T215D, K219Q, K219E, K219N, K219R, P225H, or M230L (numbering according to HXB2 numbering system). The disclosed methods can be used to detect one or more (e.g., two) of these mutant alleles of HIV-1 reverse transcriptase in a biological sample. In some embodiments, the methods can be used to detect a first allele and a second allele of HIV-1 or HIV-2, in a single qPCR assay. For example, a first allele and a second allele selected from one of K65R and M184V, K65R and M184I, K65R and K103N, K70E and M184V, K70E and M184I, K70E and K103N, K103N and M184V, or K103N and M184I.

Administration of tenofovir and emtricitabine (Truvada®) is the most commonly prescribed combination drug for HIV, with estimated annual sales of $2.4 billion dollars. Tenofovir resistance can develop by the K65R or K70E mutations, and emtricitabine resistance by M184V/I of which all involve single nucleotide changes in HIV RT gene. Accordingly, in some embodiments, the disclosed methods can be applied to assess resistant mutations to combination therapy with tenofovir and emtricitabine. Additionally, the linked HIV-1 RT K103N and Y181C mutations can confer resistance to non-nucleoside reverse transcriptase inhibitors (NNRTIs), and, in some embodiments, the disclosed methods can be applied to assess these resistant mutations to NNRTI therapy.

Exemplary oligonucleotide primers for use in the disclosed methods of detecting one or more mutant alleles of HIV-1 reverse transcriptase are provided in Table 1.

TABLE 1
Exemplary target nucleic acids and alleles, Plus primers, and control
primers for targeting alleles of HIV-1 Reverse Transcriptase

| | | | Wild-type specific | | |
|---|---|---|---|---|---|
| | | | Plus primer | | |
| | | | Control/Total | | |
| | | Allele-specific | primer sequence | Additional control | Exemplary reverse/ |
| | | Plus Primer | (with LNA at 3′end | primers (when | forward primer |
| | | sequence (com- | that is complemen- | using three or | for PCR assay (does |
| | | plementary to | tary to wild-type | four control | not need to be for |
| | Allele | mutant sequence) | allele sequence) | primers are used) | linked PCR assays) |
| a | K65R | CTCCARTATTTGC | CTCCARTATTTGC | CTCCARTATTT | TATTCCTAATTGAA |
| | | CATAAAA [symbol] + G | CATAAAA [symbol] + A | GCCATAAAA [symbol] + | CYTCCCA |
| | | (SEQ ID NO: 1) | (SEQ ID NO: 2) | C | (SEQ ID NO: 3) |
| | | CTCCAATATTTGC | CTCCAATATTTG | (SEQ ID NO: 33) | TATTCCTAATTGAA |
| | | CATAAAA [symbol] + G | CCATAAAA [symbol] + A | CTCCARTATTT | CCTCCCA |
| | | (SEQ ID NO: 39) | (SEQ ID NO: 41) | GCCATAAAA [symbol] + | (SEQ ID NO: 37) |
| | | CTCCAGTATTTGC | CTCCAGTATTTG | T | TATTCCTAATTGAA |
| | | CATAAAA [symbol] + G | CCATAAAA [symbol] + A | (SEQ ID NO: 34) | CTTCCCA |
| | | (SEQ ID NO: 40) | (SEQ ID NO: 42) | CTCCAATATTT | (SEQ ID NO: 38) |
| | | | | GCCATAAAA [symbol] + | |
| | | | | C | |
| | | | | (SEQ ID NO: 43) | |
| | | | | CTCCAATATTT | |
| | | | | GCCATAAAA [symbol] + | |
| | | | | T | |
| | | | | (SEQ ID NO: 44) | |
| | | | | CTCCAGTATTT | |
| | | | | GCCATAAAA [symbol] + | |
| | | | | C | |
| | | | | (SEQ ID NO: 45) | |
| | | | | CTCCAGTATTT | |
| | | | | GCCATAAAA [symbol] + | |
| | | | | T | |
| | | | | (SEQ ID NO: 46) | |
| b | K70E | GCCATAAAAAAG | GCCATAAAAAAG | | TATTCCTAATTGAA |
| | | AAGGACCAGTA [symbol] + | AAGGACCAGTA [symbol] + | | CYTCCCA |
| | | G | A | | (SEQ ID NO: 3) |
| | | (SEQ ID NO: 4) | (SEQ ID NO: 5) | | |
| c | M184 | CTAAGTCAGATCC | CTAAGTCAGATC | | TAGTATAAACAAT |
| | V | TACATACAAGTCA | CTACATACAAGT | | GAGACACCAGGGA |
| | | TCC [symbol] + C | CATCC [symbol] + T | | TTA |
| | | (SEQ ID NO: 6) | (SEQ ID NO: 7) | | (SEQ ID NO: 8) |
| d | M184I | CCCTATTTCTAAG | CCCTATTTCTAA | | TAGTATAAACAAT |
| | | TCAGATCCTACAT | GTCAGATCCTAC | | GAGACACCAGGGA |
| | | ACAAAGTCAT [symbol] + T | ATACAAAGTCAT | | TTA |
| | | (SEQ ID NO: 9) | [symbol] + C | | (SEQ ID NO: 8) |
| | | | (SEQ ID NO: 10) | | |
| e | K103N | CCCACATCTAGTA | CCCACATCTAGT | CCCACATCTAG | AAGTGGAGAAAAT |
| | | CTGTCACTGATT [symbol] + | ACTGTCACTGAT | TACTGTCACTG | TAGTAGATTTCAG |
| | | A | T [symbol] + T | ATT [symbol] + C | GGA |
| | | (SEQ ID NO: 11) | (SEQ ID NO: 12) | (SEQ ID NO: 13) | (SEQ ID NO: 14) |
| | | | | CCCACATCTAG | |
| | | | | TACTGTCACTG | |
| | | | | ATT/ + G | |
| | | | | (SEQ ID NO: 15) | |
| f | K103N | CCCACATCTAGTA | CCCACATCTAGT | CCCACATCTAG | AAGTGGAGAAAAT |
| | | CTGTCACTGATT [symbol] + | ACTGTCACTGAT | TACTGTCACTG | TAGTAGATTTCAG |
| | | G | T [symbol] + T | ATT [symbol] + C | GGA |
| | | (SEQ ID NO: 15) | (SEQ ID NO: 12) | (SEQ ID NO: 13) | (SEQ ID NO: 14) |
| | | | | CCCACATCTAG | |
| | | | | TACTGTCACTG | |
| | | | | ATT [symbol] + A | |
| | | | | (SEQ ID NO: 11) | |
| g | Y181C | CTACATACAAGTC | CTACATACAAGT | | CACCAGGGATTAG |
| | | ATCCATATATTG [symbol] + | CATCCATATATT | | ATATCAATATAAT |
| | | C | G [symbol] + T | | GTG |
| | | (SEQ ID NO: 16) | (SEQ ID NO: 17) | | (SEQ ID NO: 18) |
| h | G190A | CTATGTTGCCCTA | CTATGTTGCCCT | | CACCAGGGATTAG |
| | | TTTCTAAGTCAGA | ATTTCTAAGTCA | | ATATCAATATAAT |
| | | [symbol] + G | GA [symbol] + C | | GTG |
| | | (SEQ ID NO: 19) | (SEQ ID NO: 20) | | (SEQ ID NO: 18) |

In Table 1, underlined nucleic acids indicate the penultimate mismatch, and a nucleic acid following a “+” is a locked nucleic acid (LNA). In Table 1, several sequences include an “R” nucleotide, including SEQ ID NOs: 1, 2, 33, and 34. The “R” indicates that the particular position of the nucleic acid molecule can be adenine or guanine. For reference, corresponding sequences with “A” or “G” nucleotides are shown as SEQ ID NOs: 39-46. Additionally, SEQ ID NO: 3 includes a “Y” nucleic acid. The “Y” indicates that the particular position of the nucleic acid molecule can be cytosine or thymine. For reference, corresponding sequences with “C” or “T” nucleotides are shown as SEQ ID NOs: 37-38. The person of skill in the art will appreciate that amplification assay

In some embodiments of the disclosed method, the test amplification and the control amplification include use of a first plus primer, a reverse primer, and a first control primer as listed in one of the rows of Table 1. In additional embodiments, the test amplification and/or the control amplification include use of a first plus primer, a reverse primer, a first control primer, and an additional control primer as listed in one of the rows of Table 1.

In other embodiments, the method comprises detecting a K65R allele of HIV-1 reverse transcriptase, and the first plus primer, the first control primer, the reverse primer, comprise, consist, or consist essentially of, the nucleic acid sequences set forth as SEQ ID NOs: 1, 2, and 3, respectively. In these assays, SEQ ID NOs: 1 and 2 can be substituted with SEQ ID NOs: 29 and 41, or SEQ ID NOs: 40 and 42. Further, SEQ ID NO 3 can be substituted with SEQ ID NOs: 37 or 38. In some such assays, additional control primers can be added to the amplification assay, for example SEQ ID NOs: 33 and 34 can be added to the assay.

In other embodiments, the method comprises detecting a K65R allele of HIV-1 reverse transcriptase, and the first plus primer, the first control primer, the reverse primer, comprise, consist, or consist essentially of, the nucleic acid sequences set forth as SEQ ID NOs: 39 and 41, and 3 (or SEQ ID NOs: 37 or 38), respectively, and additional control primers can be included in the assay, such as SEQ ID NOs: 43 and 44.

In other embodiments, the method comprises detecting a K65R allele of HIV-1 reverse transcriptase, and the first plus primer, the first control primer, the reverse primer, comprise, consist, or consist essentially of, the nucleic acid sequences set forth as SEQ ID NOs: 40 and 42, and 3 (or SEQ ID NOs: 37 or 38), respectively, and additional control primers can be included in the assay, such as SEQ ID NOs: 45 and 46.

In other embodiments, the method comprises detecting a K65R allele and a M184V allele of HIV-1 reverse transcriptase, and the first plus primer, the first control primer, the second plus primer, and the second control primer, comprise, consist, or consist essentially of, the nucleic acid sequences set forth as SEQ ID NOs: 1, 2, 6, and 7, respectively. In further embodiments, the method comprises detecting a K65R and a M184I allele of HIV-1 reverse transcriptase, and the first plus primer, the first control primer, the second plus primer, and the second control primer, comprise, consist, or consist essentially of, the nucleic acid sequences set forth as SEQ ID NOs: 1, 2, 9, and 10, respectively. In these assays, SEQ ID NOs: 1 and 2 can be substituted with SEQ ID NOs: 29 and 41, or SEQ ID NOs: 40 and 42.

In other embodiments, the method comprises detecting a K70E allele of HIV-1 reverse transcriptase, and the first plus primer, the first control primer, the reverse primer, comprise, consist, or consist essentially of, the nucleic acid sequences set forth as SEQ ID NOs: 4, 5, and 3, respectively. In these assays, SEQ ID NO 3 can be substituted with SEQ ID NOs: 37 or 38.

In other embodiments, the method comprises detecting a M184V allele of HIV-1 reverse transcriptase, and the first plus primer, the first control primer, the reverse primer, comprise, consist, or consist essentially of, the nucleic acid sequences set forth as SEQ ID NOs: 6, 7, and 8, respectively.

In other embodiments, the method comprises detecting a M184I allele of HIV-1 reverse transcriptase, and the first plus primer, the first control primer, the reverse primer, comprise, consist, or consist essentially of, the nucleic acid sequences set forth as SEQ ID NOs: 9, 10, and 8, respectively.

In other embodiments, the method comprises detecting a K103N allele of HIV-1 reverse transcriptase, and the first plus primer, the first control primer, the reverse primer, comprise, consist, or consist essentially of, the nucleic acid sequences set forth as SEQ ID NOs: 11, 12, and 14, respectively. In some such assays, additional control primers can be added to the amplification assay, for example SEQ ID NOs: 13 and 15 can be added to the assay.

In other embodiments, the method comprises detecting a K103N allele of HIV-1 reverse transcriptase, and the first plus primer, the first control primer, the reverse primer, comprise, consist, or consist essentially of, the nucleic acid sequences set forth as SEQ ID NOs: 15, 12, and 14, respectively. In some such assays, additional control primers can be added to the amplification assay, for example SEQ ID NOs: 13 and 11 can be added to the assay.

In other embodiments, the method comprises detecting a Y181C allele of HIV-1 reverse transcriptase, and the first plus primer, the first control primer, the reverse primer, comprise, consist, or consist essentially of, the nucleic acid sequences set forth as SEQ ID NOs: 16, 17, and 18, respectively.

In other embodiments, the method comprises detecting a G190A allele of HIV-1 reverse transcriptase, and the first plus primer, the first control primer, the reverse primer, comprise, consist, or consist essentially of, the nucleic acid sequences set forth as SEQ ID NOs: 19, 20, and 18, respectively.

### B. Additional Description of the Disclosed Methods

The skilled artisan will appreciate that detecting the presence or absence (or amount or proportion) of the target nucleic acid molecule as described herein using qPCR assays can include detecting the target nucleic acid molecule after a particular amplification cycle of the qPCR assay. For example, after 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, and/or 50 amplification cycles of the qPCR assay, or at least that may cycles, or no more than that many cycles.

In several embodiments, the biological sample can be selected from any clinical samples useful for detection of disease or infection (e.g., HIV infection) or an allele of interest in a subjects. Exemplary biological samples include, without limitation, cells, cell lysates, blood smears, cytocentrifuge preparations, cytology smears, bodily fluids (e.g., blood, plasma, serum, saliva, sputum, urine, bronchoalveolar lavage, semen, CSF, etc.), tissue biopsies or autopsies, fine-needle aspirates, and/or tissue sections. In one embodiment, the biological sample is a urine sample. In another embodiment, the biological sample is a serum sample. In a further embodiment, the biological sample is a CSF sample. In a particular example, a biological sample is obtained from a subject having, suspected of having or at risk of having, HIV; for example, a subject having HIV infection. Standard techniques for acquisition of such samples are available (see, e.g. Schluger et al., J. Exp. Med. 176:1327-33, 1992; Bigby et al., Am. Rev. Respir. Dis. 133:515-18, 1986; Kovacs et al., NEJM 318:589-93, 1988; and Ognibene et al., Am. Rev. Respir. Dis. 129:929-32, 1984). The sample can be used directly or can be processed, such as by adding solvents, preservatives, buffers, or other compounds or substances. In some embodiments, nucleic acids are isolated from the sample. DNA or RNA can be extracted using standard methods. For instance, rapid DNA preparation can be performed using a commercially available kit (e.g., the Qiagen Tissue Kit, Qiagen, Inc., Valencia, Calif.). The DNA preparation technique can be chosen to yield a nucleotide preparation that is accessible to and amenable to nucleic acid amplification.

The target nucleic acid molecule can include or consist of at least about 20, 25, 30, 35, 40, 45, 50, 60, 70, 80, 90, 100, 150, 200, 250, 300, 350, 400, 450, 500, or more consecutive nucleotides of a nucleic acid sequence (such as of the HIV-1 genome, e.g., HIV-1 reverse transcriptase gene).

In some embodiments, the oligonucleotide probe can be labeled, for example with a base-linked or terminally-linked fluorophore and non-fluorescent quencher for use in qPCR assays. Fluorophores for use in qPCR assays are known in the art. They can be obtained, for example, from Life Technologies (Gaithersburg, Md.), Sigma-Genosys (The Woodlands, Tex.), Genset Corp. (La Jolla, Calif.), or Synthetic Genetics (San Diego, Calif.). Fluorophores can be conjugated to the oligonucleotides, for example by post-synthesis modification of oligonucleotides that are synthesized with reactive groups linked to bases. Useful fluorophores include: fluorescein, fluorescein isothiocyanate (FITC), carboxy tetrachloro fluorescein (TET), NHS-fluorescein, 5 and/or 6-carboxy fluorescein (FAM), 5-(or 6-) iodoacetamidofluorescein, 5-{[2(and 3)-5-(Acetylmercapto)-succinyl]amino} fluorescein (SAMSA-fluorescein), 5′-hexachloro-fluorescein (HEX), 6-carboxy-4′,5′-dichloro-2′,7′dimethoxyfluorescein,succinimidyl ester (JOE) and other fluorescein derivatives, rhodamine, Lissamine rhodamine B sulfonyl chloride, Texas red sulfonyl chloride, 5 and/or 6 carboxy rhodamine (ROX) and other rhodamine derivatives, coumarin, 7-amino-methyl-coumarin, 7-Amino-4-methylcoumarin-3-acetic acid (AMCA), and other coumarin derivatives, BODIPY fluorophores, Cascade Blue fluorophores such as 8-methoxypyrene-1,3,6-trisulfonic acid trisodium salt, Lucifer yellow fluorophores such as 3,6-Disulfonate-4-amino-naphthalimide, phycobiliproteins derivatives, Alexa fluor dyes (available from Molecular Probes, Eugene, Oreg.) and other fluorophores known to those of skill in the art. For a general listing of useful fluorophores, see also Hermanson, G. T., BIOCONJUGATE TECHNIQUES (Academic Press, San Diego, 1996).

Quenchers for use in qPCR assays are also known in the art and include, for example, 6-carboxytetramethylrhodamine,succinidyl ester (6-TAMRA; TAMRA) and “non-fluorescent quencher (NFP)” for use with TAQMAN™ probes available from Life technologies.

Several embodiments include the use of PCR and/or qPCR. PCR reaction conditions typically include either two or three step cycles. Two step cycles have a denaturation step followed by a hybridization/elongation step. Three step cycles include a denaturation step followed by a hybridization step during which the primer hybridizes to the strands of DNA, followed by a separate elongation step. The polymerase reactions are incubated under conditions in which the primers hybridize to the target sequences and are extended by a polymerase. The amplification reaction cycle conditions are selected so that the primers hybridize specifically to the target sequence and are extended.

Primers for the disclosed assays are typically designed so that all of the primers participating in a particular reaction have melting temperatures that are within at least five degrees Celsius, and more typically within two degrees Celsius of each other. Primers are further designed to avoid priming on themselves or each other. Primer concentration should be sufficient to bind to the amount of target sequences that are amplified so as to provide an accurate assessment of the quantity of amplified sequence. Those of skill in the art will recognize that the amount of concentration of primer will vary according to the binding affinity of the primers as well as the quantity of sequence to be bound. Typical primer concentrations will range from 0.01 μM to 0.5 μM.

In a typical PCR cycle, a sample including a DNA polynucleotide and a PCR reaction cocktail is denatured by treatment in thermal cycler at about 90-98° C. for 10-90 seconds. The denatured polynucleotide is then hybridized to oligonucleotide primers by treatment in a thermal cycler at a temperature of about 30-65° C. for 1-2 minutes. Chain extension then occurs by the action of a DNA polymerase on the polynucleotide annealed to the oligonucleotide primer. This reaction occurs at a temperature of about 70-75° C. for 30 seconds to 5 minutes. Any desired number of PCR cycles may be carried out depending on variables including but not limited to the amount of the initial DNA polynucleotide, the length of the desired product and primer stringency. The above temperature ranges and the other numbers are exemplary and not intended to be limiting. These ranges are dependent on other factors such as the type of enzyme, the type of container or plate, the type of biological sample, the size of samples, etc. One of ordinary skill in the art will recognize that the temperatures, time durations and cycle number can readily be modified as necessary.

Several embodiments include quantitative real-time polymerase chain reaction (qPCR), which is used to simultaneously quantify and amplify a specific part of a given nucleic acid molecule. It is used, for example, to determine whether or not a specific sequence is present in the sample; and if it is present, the number of copies in the sample.

qPCR monitors the fluorescence emitted during the reaction as an indicator of amplicon production during each PCR cycle, as opposed to endpoint detection. The real-time progress of the reaction can be viewed in some systems. Typically, qPCR uses the detection of a fluorescent reporter. Typically, the fluorescent reporter's signal increases in direct proportion to the amount of PCR product in a reaction. By recording the amount of fluorescence emission at each cycle, it is possible to monitor the PCR reaction during exponential phase where the first significant increase in the amount of PCR product correlates to the initial amount of target template. The higher the starting copy number of the nucleic acid target, the sooner a significant increase in fluorescence is observed. Thus, the procedure follows the general pattern of polymerase chain reaction, but the nucleic acid molecule is quantified after each round of amplification. In several embodiments the amplified nucleic acid molecule is quantified by the use of fluorescent dye that intercalates with double-strand DNA. In other embodiments (e.g., when multiplex qPCR assays are utilized) amplified nucleic acid molecule is quantified by use of oligonucleotide probes labeled with a reporter fluorophore that can be detected in the qPCR assay.

In certain embodiments, the amplified products are directly visualized with detectable label such as a fluorescent DNA-binding dye. In one embodiment the amplified products are quantified using an intercalating dye, including but not limited to SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin. For example, a DNA binding dye such as SYBR green binds double stranded DNA and an increase in fluorescence intensity can be measured. For example, the fluorescent dsDNA dye can be added to the buffer used for a PCR reaction. The PCR assay can be performed in a thermal cycler, and after each cycle, the levels of fluorescence are measured with a detector, such as a camera. The dye fluoresces much more strongly when bound to dsDNA (e.g., amplified PCR product). Because the amount of the dye intercalated into the double-stranded DNA molecules is typically proportional to the amount of the amplified DNA products, the amount of amplified nucleic acid can be quantified by detecting the fluorescence of the intercalated dye using detection instruments known in the art. When referenced to a standard dilution, the dsDNA concentration in the PCR can be determined.

In addition to various kinds of fluorescent DNA-binding dye, other luminescent labels such as sequence specific oligonucleotide probes can be employed in the amplification reaction to facilitate the detection and quantification of the amplified product. Probe based quantitative amplification relies on the sequence-specific detection of a desired amplified product. Unlike the dye-based quantitative methods, it utilizes target-specific probe labeled with a detectable marker such as a base-linked or terminally-linked fluorophore and quencher. Such markers are known to the person of ordinary skill in the art and described herein. Further, methods for performing probe-based quantitative amplification are well established in the art (see, e.g., U.S. Pat. No. 5,210,015).

For detection using oligonucleotide probes, the reaction is prepared as usual for PCR conditions, with the addition of the sequence specific labeled oligonucleotide probe. After denaturation of the DNA, the labeled probe is able to bind to its complementary sequence in the region of interest of the template DNA. When the PCR reaction is heated to the proper extension temperature, the polymerase is activated and DNA extension proceeds. As the polymerization continues it reaches the labeled probe bound to the complementary sequence of DNA. The polymerase breaks the probe into separate nucleotides, and separates the fluorescent reporter from the quencher. This results in an increase in fluorescence as detected by the optical assembly. As PCR cycle number increases more and more of the fluorescent reporter is liberated from its quencher, resulting in a well-defined geometric increase in fluorescence. This allows accurate determination of the final, and initial, quantities of DNA.

In one embodiment, the fluorescently-labeled probes (such as probes disclosed herein) rely upon fluorescence resonance energy transfer (FRET), or in a change in the fluorescence emission wavelength of a sample, as a method to detect hybridization of a DNA probe to the amplified target nucleic acid in real-time. For example, FRET that occurs between fluorogenic labels on different probes (for example, using HybProbes) or between a donor fluorophore and an acceptor or quencher fluorophore on the same probe (for example, using a molecular beacon or a TAQMAN™ probe) can identify a probe that specifically hybridizes to the DNA sequence of interest. In some embodiments, the fluorescently-labeled DNA probes used to identify amplification products have spectrally distinct emission wavelengths, thus allowing them to be distinguished within the same reaction tube, for example in multiplex PCR, such as a multiplex qPCR.

Any type of thermal cycler apparatus can be used for the amplification of, for example, HIV nucleic acids, as described above and/or the determination of hybridization. Examples of suitable apparatuses include PTC-100® Peltier Thermal Cycler (MJ Research, Inc.; San Francisco, Calif.), a ROBOCYCLER® 40 Temperature Cycler (Agilent/Stratagene; Santa Clara, Calif.), or GeneAmp® PCR System 9700 (Applied Biosystems; Foster City, Calif.). For qPCR, any type of real-time thermocycler apparatus can be used. For example, ICYCLER IQ™ or CFX96™ real-time detection systems (Bio-Rad, Hercules, Calif.), LIGHTCYCLER® systems (Roche, Mannheim, Germany), a 7700 Sequence Detector (Perkin Elmer/Applied Biosystems; Foster City, Calif.), ABI™ systems such as the 7000, 7300, 7500, 7700, 7900, or ViiA7 systems (Applied Biosystems; Foster City, Calif.), or an MX4000™, MX3000™ or MX3005™ qPCR system (Agilent/Stratagene; Santa Clara, Calif.), DNA ENGINE OPTICON® Continuous Fluorescence Detection System (Bio-Rad, Hercules, Calif.), ROTOR-GENE® Q real-time cycler (Qiagen, Valencia, Calif.), or SMARTCYCLER® system (Cepheid, Sunnyvale, Calif.) can be used to amplify and detect nucleic acid sequences in real-time. In some embodiments, qPCR is performed using a TAQMAN® array format, for example, a microfluidic card in which each well is pre-loaded with primers and probes for a particular target. The reaction is initiated by adding a sample including nucleic acids and assay reagents (such as a PCR master mix) and running the reactions in a real-time thermocycler apparatus.

### III. Isolated Nucleic Acid Molecules and Compositions Comprising Same

Isolated oligonucleotide primers (which as defined herein also include the complementary sequence and corresponding RNA sequences) for use in the disclosed methods, and compositions comprising such primers, are provided herein.

The isolated oligonucleotide primers can comprise or consist of at least 10 consecutive nucleotides (such as at least 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 consecutive nucleotides) from a target nucleic acid sequence (e.g., an HIV-1 sequence). For example, in some embodiments, the isolated oligonucleotide primers can include or consist of 10-50 nucleotides, such as, 12-15, 12-20, 12-25, 12-30, 12-35, 12-40, 12-45, 12-50, 14-15, 14-16, 14-18, 14-20, 14-25, 14-30, 15-16, 15-18, 15-20, 15-25, 15-30, 15-35, 15-40, 15-45, 15-50, 16-17, 16-18, 16-20, 16-22, 16-25, 16-30, 16-40, 16-50, 17-18, 17-20, 17-22, 17-25, 17-30, 18-19, 18-20, 18-22, 18-25, 18-30, 19-20, 19-22, 19-25, 19-30, 20-21, 20-22, 20-25, 20-30, 20-35, 20-40, 20-45, 20-50, 21-22, 21-25, 21-30, 22-25, 22-30, 22-40, 22-50, 23-24, 23-25, 23-30, 24-25, 24-30, 25-30, 25-35, 25-40, 25-45, or 25-50 consecutive nucleotides from a target nucleic acid sequence.

In some embodiments, any of the probes or primers disclosed herein can be of a maximum length, for example no more than 15, 25, 25, 40, 50, 75, 100, or 150 nucleotides in length. Any of the isolated nucleic acid sequences disclosed herein may consist or consist essentially of the disclosed sequences, or include nucleic acid molecules that have a maximum length of 15, 20, 25, 30, 35, 40, 45, 50, 55, 60, 65, 70, 75 or 80 contiguous nucleotides of the disclosed sequence. The disclosed contiguous sequences may also be joined at either end to other unrelated sequences.

In some embodiments, the oligonucleotide primers comprise or consist of the sequence of any one of the primers listed herein, such as a primer listed in Table 1 above. These oligonucleotides can be employed as effective oligonucleotide primers for amplification and/or detection of target nucleic acid molecule sequences.

In some embodiments, the isolated nucleic acid molecule comprises a plus primer comprising or consisting of the nucleic acid sequence set forth as any one of SEQ ID NOs: 1-2, 4-5, 6-7, 9-12, 15-17, 19-20, or 39-42.

Compositions comprising one or more of the probes or primers disclosed herein are also provided, and are useful, for example, in the disclosed methods.

In some embodiments, the composition can include a primer pair comprising a forward and a reverse primer for amplifying a target nucleic acid molecule comprising a mutant first allele, wherein the forward primer is a first plus primer comprising a locked nucleic acid at the 3′ end that is complementary to the mutant first allele of the target nucleic acid molecule, a mismatch nucleotide at the −1 position from the 3′ end that is not complementary to the target nucleic acid molecule, and remaining nucleotides that are complementary to the target nucleic acid molecule. The composition can further include a first control primer comprising a locked nucleic acid at the 3′ end that is complementary to a wildtype first allele corresponding to the mutant first allele, and remaining nucleotides that are the same as the first plus primer. In some such embodiments, the composition is useful in methods for detecting a mutant allele of HIV-1 reverse transcriptase, and the first plus primer, the first control primer, and the reverse primer, comprise or consist of the nucleic acid sequences set forth as one of: SEQ ID NOs: 1, 2, and 3, respectively, SEQ ID NOs: 29, 41, and 3, respectively; SEQ ID NOs: 40, 42, and 3, respectively, SEQ ID NOs: 1, 2, and 37, respectively, SEQ ID NOs: 29, 41, and 37, respectively, SEQ ID NOs: 40, 42, and 37, respectively, SEQ ID NOs: 1, 2, and 38, respectively, SEQ ID NOs: 29, 41, and 38, respectively, SEQ ID NOs: 40, 42, and 38, respectively, SEQ ID NOs: 4, 5 and 3, respectively, SEQ ID NOs: 4, 5, and 37, respectively, SEQ ID NOs: 4, 5, and 38, respectively, SEQ ID NOs: 6, 7, and 8, respectively, SEQ ID NOs: 9, 10, and 10, respectively, SEQ ID NOs: 11, 12, and 14, respectively, SEQ ID NOs: 15, 12, and 14, respectively, SEQ ID NOs: 16, 17, and 17, respectively, or SEQ ID NOs: 19, 20, and 20, respectively.

In some embodiments, the composition can include a primer pair comprising a forward and a reverse primer for amplifying a target nucleic acid molecule comprising a mutant first allele and a mutant second allele, wherein the forward primer is a first plus primer comprising a locked nucleic acid at the 3′ end that is complementary to the mutant first allele of the target nucleic acid molecule, a mismatch nucleotide at the −1 position from the 3′ end that is not complementary to the target nucleic acid molecule, and remaining nucleotides that are complementary to the target nucleic acid molecule, and the reverse primer of the test primer pair is a second plus primer comprising a locked nucleic acid at the 3′ end that is complementary to the mutant second allele, a mismatch nucleotide at the −1 position from the 3′ end that is not complementary to the target nucleic acid molecule, and remaining nucleotides that are complementary to the target nucleic acid molecule. The composition can further include a first control primer comprising a locked nucleic acid at the 3′ end that is complementary to a wildtype first allele corresponding to the mutant first allele, and remaining nucleotides that are the same as the first plus primer, and a second control primer comprising a locked nucleic acid at the 3′ end that is complementary to a wildtype second allele corresponding to the mutant second allele, and remaining nucleotides that are the same as the second plus primer. In some such embodiments, the composition is useful in methods for detecting first and second mutant alleles of HIV-1 reverse transcriptase, and the first plus primer, the first control primer, the second plus primer, and the second control primer, comprise or consist of the nucleic acid sequences set forth as SEQ ID NOs: 1, 2, 6, and 7, respectively, or SEQ ID NOs: 1, 2, 9, and 10, respectively.

The isolated nucleic acid molecules and/or compositions disclosed herein can be supplied in the form of a kit for use in an assay to identify or characterize a target nucleic acid molecule. In such a kit, an appropriate amount of one or more of the primers disclosed herein, are provided in one or more containers. A nucleic acid probe may be provided suspended in an aqueous solution or as a freeze-dried or lyophilized powder, for instance. The container(s) in which the nucleic acid(s) are supplied can be any conventional container that is capable of holding the supplied form, for instance, microfuge tubes, ampoules, or bottles. Control reagents, such as control nucleic acid molecules can also be included.

In some examples, one or more sets of primers, may be provided in pre-measured single use amounts in individual, typically disposable, tubes or equivalent containers. With such an arrangement, the sample to be tested for the presence of the target nucleic acids can be added to the individual tube(s) and amplification carried out directly.

The amount of nucleic acid probe supplied in the kit can be any appropriate amount, and may depend on the target market to which the product is directed. For instance, if the kit is adapted for research or clinical use, the amount of each nucleic acid primer provided would likely be an amount sufficient to prime several detection reactions.

In some embodiments, kits also may include the reagents necessary to carry out ASPCR assays, including sample preparation reagents, appropriate buffers, salts, tubes or assay cells. In other particular embodiments, the kit includes equipment, reagents, and instructions for extracting and/or purifying nucleic acid molecules from a sample.

### EXAMPLE

The following example is provided to illustrate particular features of certain embodiments, but the scope of the claims should not be limited to those features exemplified.

### Example 1

### Allele Specific PCR Assay for the Detection of HIV-1 Minor Variants and Linked Drug Resistance Mutations

This example illustrates a novel Allele-Specific PCR (ASPCR) assay that provides improved specificity and sensitivity compared to known assays for detecting variations in a target nucleic acid sequence. Standard ASPCR serves as a good alternative to standard genotypic and phenotypic HIV-Drug Resistance (HIVDR) assays, addressing limitations associated with these assays, such as low sensitivity and high cost. Even though ASPCR is used in research settings, routine use in the clinic has been precluded due to issues associated with: demand of high stringency conditions, vulnerability to HIV polymorphism, PCR artifacts, and detection of one mutation at a time. This example describes an improved ASPCR assay that addresses the above issues. Using primers that carry both a penultimate mismatch and a 3′ Locked Nucleic Acid (LNA), the discriminatory power of ASPCR between mutant and wild type templates was increased. Additionally, by introducing a new method for normalization, which is based on the delta Ct (ΔCt) between the allelic specific reaction and a reaction that uses degenerative primers at the 3′ end for the studied mutation, issues with HIV polymorphism were addressed. These modifications generated a more robust assay. In addition, by replacing the use of Taq polymerase during the first round amplification with a 3^{rd} generation proof reading polymerase, the background of the assay was reduced, and associated PCR artifacts were eliminated. Finally, using an allele specific primer for both the forward and the reverse primer, it was possible to detect two mutations in one reaction, and determine their linkage. The improved ASPCR assay can serve as a simplified method for surveillance and monitoring of HIV drug resistance.

### INTRODUCTION

In several clinical studies, HIV drug-resistance (HIVDR) testing has been associated with better patient management, improved virological and clinical outcomes, and better overall survival (Palella et al., Ann. Intern. Med. 151:73-84, 2009; Cortez et al., Viruses 3:347-378. doi:10.3390/v3040347, 2011). HIVDR testing is recommended for selection and optimization of antiretroviral (ARV) therapy in patient management, as summarized in the DHHS Antiretroviral Guidelines for the Treatment of Adult and Adolescents HIV infections (Guidelines for the Use of Antiretroviral Agents in HIV-1-Infected Adults and Adolescents. 2013. (aidsinfo.nih.gov/guidelines)). HIV drug resistance assays are either phenotypic or genotypic based. Phenotypic assays use the generation of recombinant viruses that subsequently are tested for growth in Drug Susceptibility Assays against varying drug concentrations (Hertogs et al., Antimicrob. Agents Chemother. 42:269-276, 1998; Petropoulos et al., Antimicrob. Agents Chemother 44:920-928, 2000). Genotypic assays involve sequencing or base point detection of known mutations that confer drug resistance. There are two commercially and FDA-approved HIV-1 genotyping tests, the TruGene genotyping assay (Grant et al., J. Clin. Microbiol. 41:1586-1593, 2003) and the ViroSeq HIV-1 genotyping system (Eshleman et al., J. Clin. Microbiol. 43:813-817, 2005), and several “in house” assays, that are performed by reference laboratories for HIVDR and surveillance.

A major limitation of standard HIV drug resistance assays is the lack of sensitivity to detect low-frequency drug-resistance variants that are present at a frequency of less than 20% (Church et al., J. Mol. Diag. 8:430-432, 2006; Tsiatis et al., J. Mol. Diagn. 12:425-432, 2010). As per DHHS guidelines, HIVDR testing is recommended to be performed while patient is under ARV therapy or during a short period after treatment interruption, as without the drug-selection pressure the wild-type virus quickly becomes the dominant population, dropping resistant variants below the detection limit (Guidelines for the Use of Antiretroviral Agents in HIV-1-Infected Adults and Adolescents. 2013. (aidsinfo.nih.gov/guidelines)). Failure to detect these minority resistant variants can result in poor clinical treatment outcomes. The importance of these minority variants has been best demonstrated in women that received single dose nevirapine, where the presence of low frequency mutations was associated with subsequent nevirapine containing regimen failure (Jourdain et al., N. Engl. J. Med. 351:229-240, 2004; Boltz et al., PNAS. 22:9202-9207, 2011).

For the detection of these minority variants, several assays with increased sensitivity have been developed, but have not entered routine clinical testing. Single Genome Sequencing (SGS) and Ultra-Deep Pyrosequencing (UDPS) offer an advantage over genotyping or detecting minor variants; however, their high cost, the need for IT support as in the case for UDPS or the high labor demands of SGS, have limited their routine use (Palmer et al., J. Clin. Microbiol. 43:406-413, 2005; Margulies et al., Nature. 15:376-380, 2005; Wang et al., Genome Res. 17:1195-1201, 2007). Several other methods have been developed with lower cost including oligoligase assay (OLA), LigAMP, allele-specific or mutation-specific real time PCR (ASPCR), and parallel allele-specific sequencing (PASS) (Villahermosa et al., J Clin Microbiol. 4:238-248, 2001; Shi et al., Nat. Methods. 1: 141-147, 2004; Little et al., Curr. Protoc. Hum. Genet. 2001. Chapter 9: Unit 9.8. doi:10.1002/0471142905.hg0908s07, 2001; Palmer et al., PNAS 103:7094-7099, 2006; Cai et al., Nat. Methods. 4: 123-125, 2007).

ASPCR is the most common method used for the detection of minority variants, characterized by increased sensitivity and low cost, but its use is limited to the research setting. According to Johnson et al., the main reasons that ASPCR has not entered into clinical practice is attributed to; i) the stringent conditions that are required to ensure accuracy, ii) the ability to target only one mutation at a time, iii) the absence of detecting linkage, iv) PCR artifacts that can result in false positives, and v) the presence of polymorphisms throughout the HIV genome that can affect primer binding and require the use of subtype-specific primers (Johnson et al., J. Antimicrob. Chemother. 65:1322-1326). A number of assays have been developed to address these issues, specifically those associated with sensitivity and linkage. One of these assays combines the sensitivity of ASPCR with mutation-specific amplicon sequencing, allowing detection of linked drug resistance mutations (Johnson et al., PLoS One 2: e638, 2007. doi:10.1371/journal.pone.0000638), and recently a multiplex allele-specific (MAS) assay utilizing 45 allele-specific reactions using primers tagged with oligonucleotide multiplex identifiers that can bind to microspheres that are analyzed with a suspension array system (Zhang et al., J. Clin. Microbiol. 51:3666-3674, 2013).

This example describes a novel ASPCR assay that provides increase sensitivity and specificity compared to prior assays. The new assay addresses the above issues, which could potentially allow ASPCR to enter the clinic either alone or in combination with a multiplexing technology, as a sensitive HIV-DR assay for patient care, surveillance, and monitoring. Furthermore the new assay expands applications of ASPCR to address linked drug resistance mutations that are important for combination antiretroviral therapy (cART) and could allow for the development of therapy tailored sensitive diagnostic assays.

### Materials and Methods

Viruses and Viral Mixtures.

Viral stocks with wild-type 184V (AGT) or mutant 184V (GTG) were generated by CaPO4 transfections into 293T cells using infectious plasmid clones of HW-1_{LAI}. Defined virus mixtures at varying ratios (0, 0.01, 0.1, 0.4, 1, 2, 5, 10, 25, 50, and 100% mutant) were prepared in HIV-seronegative human plasma using the viral stocks. Viral mixtures were generated to a final average copy number of 2.6×10⁵ copies/ml as determined by Amplicor HIV-1 Monitor Assay (Roche; Indianapolis, Ind.), stored at −80° C., and tested as a blinded panel.

Clinical Specimens.

All participants provided written informed consent and testing was approved by Institutional Review Board of the University of Pittsburgh.

Generation of HIV-1 subtype C standards.

Standards were generated from a cloned pro/pol subtype C 2.2 kb fragment (GenBank accession no AF286227) into pTriAmp plasmid from the HIV-1 strain 97ZA012 from South Africa. To this plasmid the K65R (AAG→AGG), K70E (AAG→GAG), M184V (ATG→GTG), M184I (ATG→ATA), or the double K65R/M184V mutations were introduced using the QuickChange II XL Site-Directed Mutagenesis kit (Statagene; La Jolla, Calif.). From these plasmids a 636 bp DNA fragment was amplified using the Phusion Hot Start II High Fidelity kit (Thermo Scientific; Waltham, Mass.) and gel purified using a DNA isolation kit (Qiagen; Venlo, Limburg, Holland). For comparative purposes, Phusion was substituted by AmpliTaq Gold (Life Technologies; Carlsbad, Calif.).

RNA Extraction and First Round Amplification.

Virus was pelleted from 500 μl of plasma by centrifugation at 24,000×g for 1 hr at 4° C. Supernatants were removed, the pellets were resuspended in 100 μl of 3M GuHCl (Fisher Scientific: Waltham, Mass.) with Proteinase K (100 μg/ml) (Ambion: Grand Island, N.Y.), and incubated at 42° C. for 1 hr to digest the virions. Subsequently 400 μl of 6M GuSCN (Sigma-Aldrich; St. Louis. Mo.) containing 200 μg of glycogen were added, and viral RNA was pelleted with 500 μl of isopropanol following centrifugation at 21,000×g for 15 min. The pellet was washed with 70% EtOH, dried, and resuspended in 30 μl of 5 mM Tris pH 7.8, containing 1 mM DTT (Fisher Scientific; Waltham, Mass.), and 1 U/μl of RNasin (Fisher Scientific; Waltham, Mass.). First round amplification from cDNA was performed utilizing subtype C specific primers, 5′-AAACAATGGCCATTGACAGAAGA-3′ forward (SEQ ID NO: 21), and 5′-GTTCATACCCCATCCAAAGAAATG-3′ reverse (SEQ ID NO: 22). For the PCR, either AmpliTaq Gold (Life Technologies) or Phusion Hot Start II High Fidelity (Thermo Scientific) was used and for comparison reasons. In order to quantify the number of cDNA copies that the amplified PCR derived from, we run a real time PCR reaction using SYBR green that compared to dsDNA standards that multiplied by 2 to calculate ssDNA copies. The amplification conditions using Phusion were 98° C. for 10 seconds, 49° C. for 20 seconds, and 72° C. for 40 seconds for a total of 40 cycles.

Primers for Allele Specific PCR.

The ASPCR primers for the detection of K65R in subtype C HIV-1 virus are; K65R forward 5′-CTCCARTATTTGCCATAAAACG-3′ (SEQ ID NO: 23, PEN), 5′-CTCCARTATTTGCCATAAAAA+G-3′ (SEQ ID NO: 24, LNA) or 5′-CTCCARTATTTGCCATAAAAC+G-3′ (SEQ ID NO: 1, PLUS), K65WT forward 5′-CTCCARTATTTGCCATAAAACA-3′ (SEQ ID NO: 25, PEN), 5′-CTCCARTATTTGCCATAAAAA+A-3′ (SEQ ID NO: 26, LNA) or 5′-CTCCARTATTTGCCATAAAAC+A-3′ (SEQ ID NO: 2, PLUS), and a common reverse primer K65REV 5′-TATTCCTAATTGAACYTCCCA-3′ (SEQ ID NO: 3). For calculation of total copies the K65total 5′-CTCCARTATTTGCCATAAAAA-3′ (SEQ ID NO: 35) was used.

For the detection of M184V the following primers were used; M184V reverse 5′-CTAAGTCAGATCCTACATACAAGTCATCCCC-3′ (SEQ ID NO: 27, PEN) or 5′-CTAAGTCAGATCCTACATACAAGTCATCCC+C-3′ (SEQ ID NO: 6, PLUS), M184V/WT reverse 5′-CTAAGTCAGATCCTACATACAAGTCATCCCT-3′ (SEQ ID NO: 28, PEN) or 5′-CTAAGTCAGATCCTACATACAAGTCATCCC+T-3′ (SEQ ID NO: 7, PLUS), and a common forward primer M184FW common primer 5′-TAGTATAAACAATGAGACACCAGGGATTA-3′ (SEQ ID NO: 8). For the detection of M184I the following primers were used; M184I reverse 5′-CCCTATTTCTAAGTCAGATCCTACATACAAGTCATGT-3′ (SEQ ID NO: 29, PEN) or 5′-CCCTATTTCTAAGTCAGATCCTACATACAAGTCATG+T-3′ (SEQ ID NO: 9, PLUS), M184I/WT reverse 5′-CCCTATTTCTAAGTCAGATCCTACATACAAGTCATGC-3′ (SEQ ID NO: 30, PEN) or 5′-CCCTATTTCTAAGTCAGATCCTACATACAAGTCATG+C-3′ (SEQ ID NO: 10, PLUS), and the M184FW (SEQ ID NO: 8) as a common forward primer. For calculation of total copies for both M184V and M184I, the M184 total primer 5′-CTATTTCTAAGTCAGATCCTACATACAAGTCATC-3′ (SEQ ID NO: 31) was used.

Primers containing locked nucleic acids were ordered from Exiqon (Woburn, Mass.). All primers were PAGE purified to avoid mispriming and false amplifications, and used at 300 nM concentration for the allele specific PCR reactions.

Allele Specific PCR.

Amplified virus template from first round amplification of clinical samples or standards was diluted down to 10⁶ copies/μl and 5×10⁶ copies were analyzed by ASPCR for the detection of the specific allele of interest. Each sample was run in 25 μl reactions in triplicate using SYBR Green Core Reagents (Life Technologies; Carlsbad, Calif.) containing 2.5 μl of 10λ SYBR Green, 1 μl of 12.5 mM dNTPs with UTP, 3.5 μl of 25 mM MgCl₂, 0.75 μl of Upper Primer, 0.75 μl of Lower Primer, 0.25 μl of 5 U/μl AmpliTaq Gold (Applied Biosystems; Foster City, Calif.), and 5 μl of first round template (10⁶ copies/O. qPCR was performed in either a ViiA7 (Applied Biosystems; Foster City, Calif.) or CFX-96 (BioRad; Hercules, Calif.) real-time thermal cycler. The amplification conditions were, 95° C. for 12 min hot-start incubation, 95° C. for 15 seconds, 55° C. for 20 seconds, and 69° C. for 1 minute for a total of 50 cycles.

Data Analysis.

Standard curves, based on the ΔCts between the allele specific reaction and the total or mixed primer reaction, were generated for half log serial dilutions of mutants in wild-type background from 100% mutant down to 0.001%. Fitting was performed using XLfit, a fitting software for Excel (IDBS). Percent mutant present in samples was calculates based on their ΔCt and the corresponding fitted standard curve.

### Results

Increased Discriminatory Power with PLUS Primers for ASPCR.

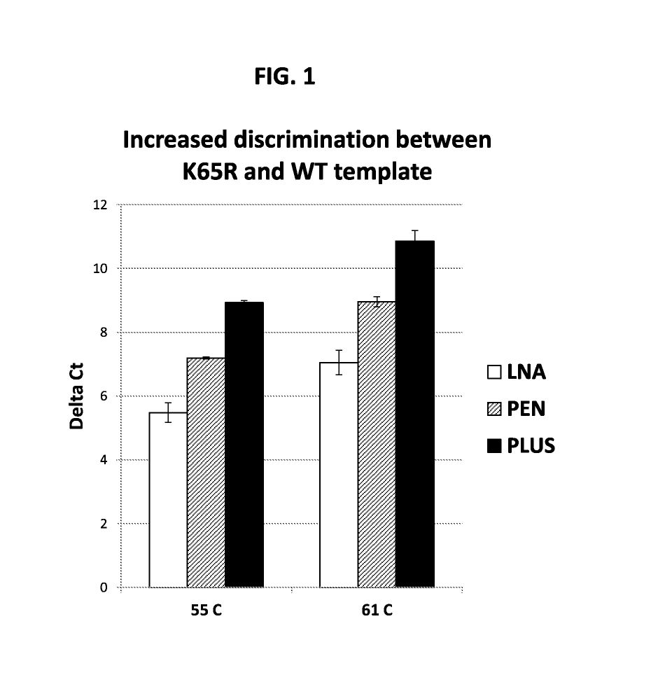

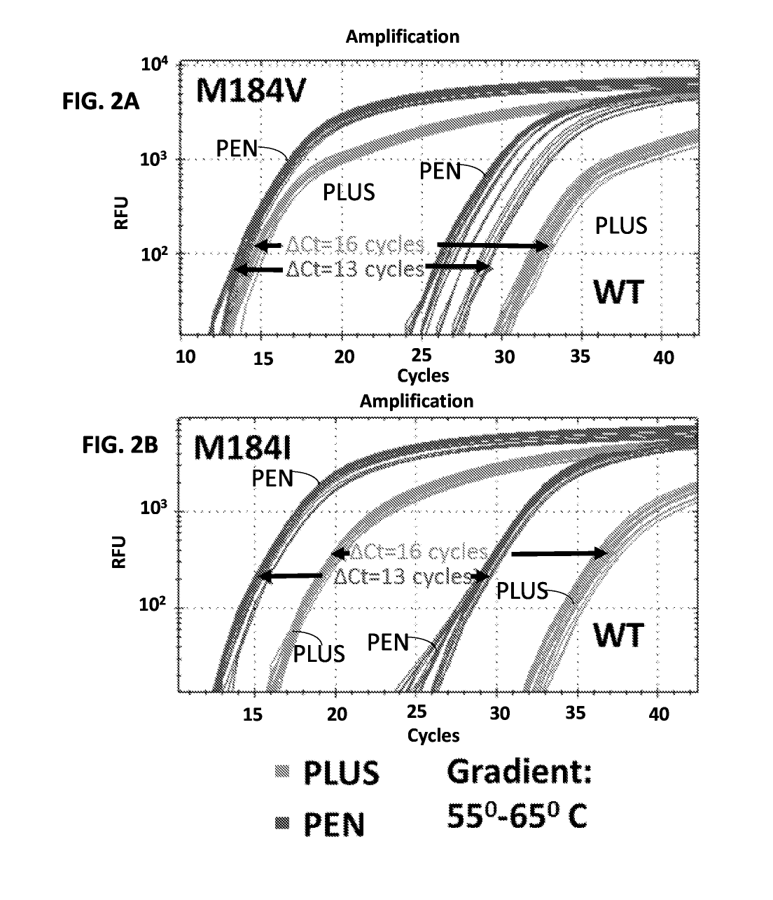

Three primer designs were tested for the development of an ASPCR assay for the detection of the K65R mutation in subtype C HIV-1 virus. The first includes the introduction of a locked nucleic acid (LNA) at the 3′ position of the allele specific primer, while the second introduces a mismatch at the penultimate site (PEN), the −1 position from the 3′ end. These designs were compared to a third, including both modifications in one AS primer designated as PLUS, for their ability to discriminate between wild type and mutant templates by ASPCR. FIG. 1 shows the differences in amplification efficiencies, expressed as a ΔCt between the 65R and the 65K template. ASPCR was performed at two different annealing temperatures of 55° C. and 61° C., respectively. Under both conditions the PLUS primer exhibited an increased ΔCt by 2 cycles when compared to the PEN primer (ΔCt of 8.9 vs. 7.2 at 55° C. and ΔCt of 10.9 vs 8.9 at 61° C.). In contrast, the LNA primer was less efficient than both the PLUS and PEN primers, regardless of conditions used (55° C. vs. 61° C.). In order to confirm that the increased discriminatory power of the PLUS primer design can increase the discriminatory power of the ASPCR, and was not unique for the K65R assay, both PLUS and PEN allele specific primers of identical sequence were also synthesized for the M184V and M184I mutant alleles of HIV-1 reverse transcriptase gene. Both primer designs were tested at gradient annealing temperatures ranging from 55° C.-65° C. against the wild type and the mutant 184V or the 184I templates. As depicted, the PLUS primer design increased the ΔCt of the ASPCR for M184V (FIG. 2A) by 3 cycles (16 vs. 13) at 65° C. and by 5 cycles (16 vs. 11) at 55° C. when compared to the corresponding PEN design. The improvement in discrimination was also observed for the M184I template. This was characterized by an increase of the ΔCt by 3 cycles (16 vs. 13) (FIG. 2B) across all temperatures tested in the gradient, confirming the increased discriminatory power of the PLUS design.

New Method for ASPCR Normalization.

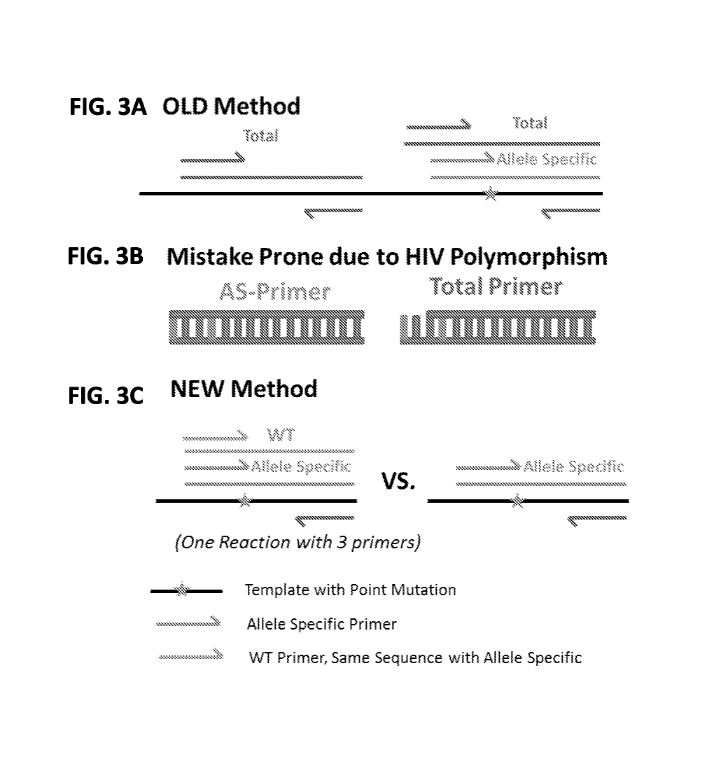

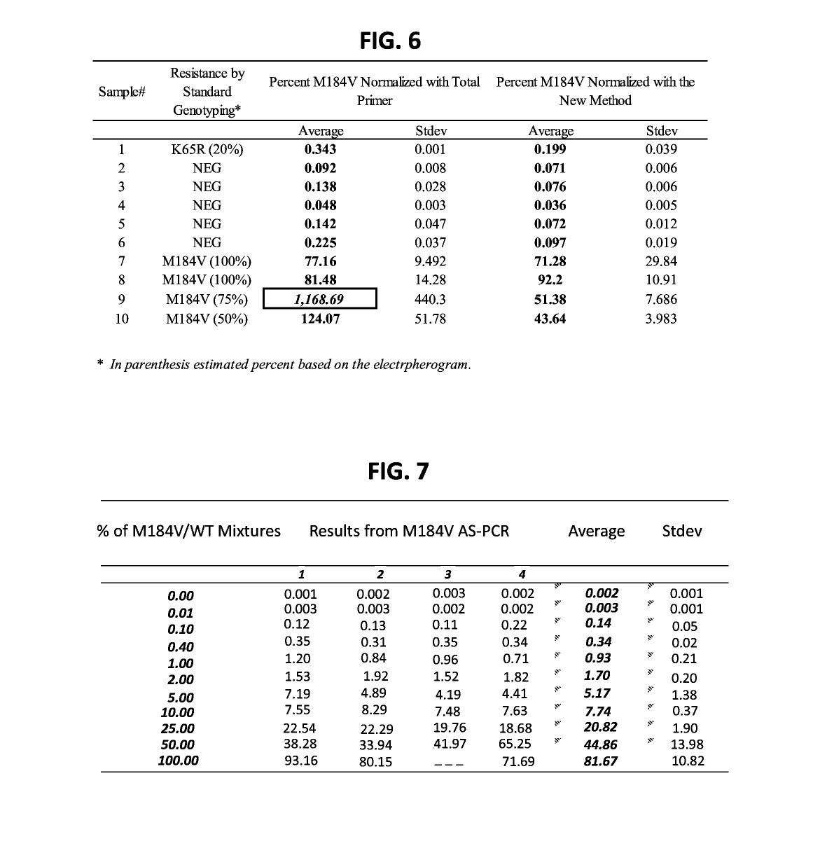

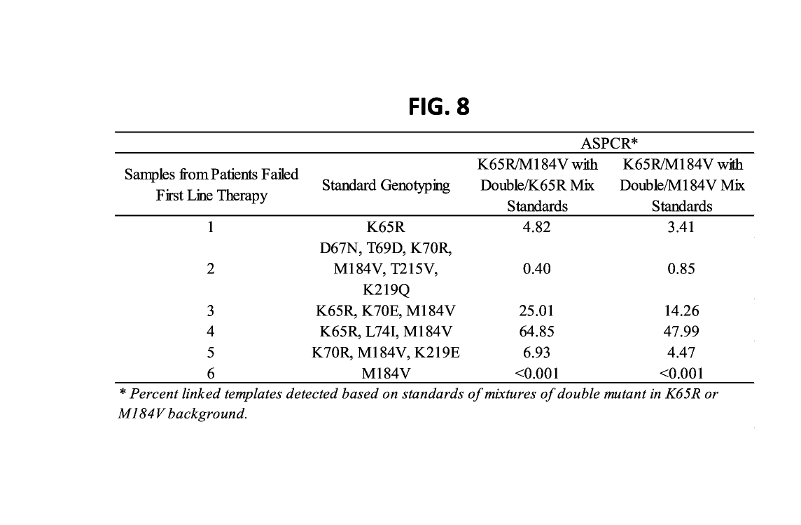

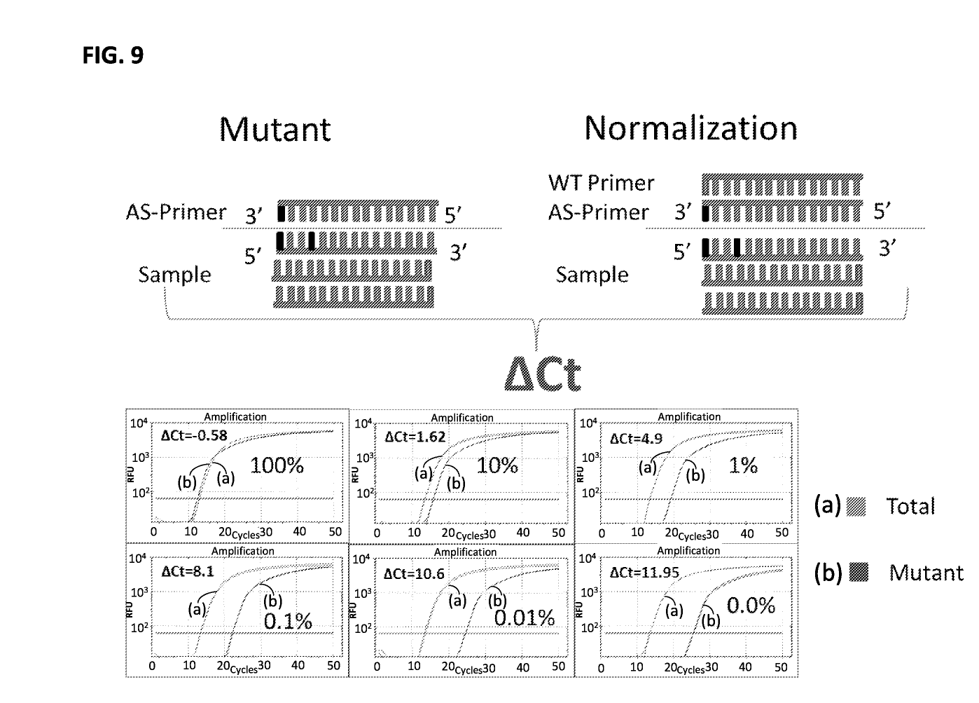

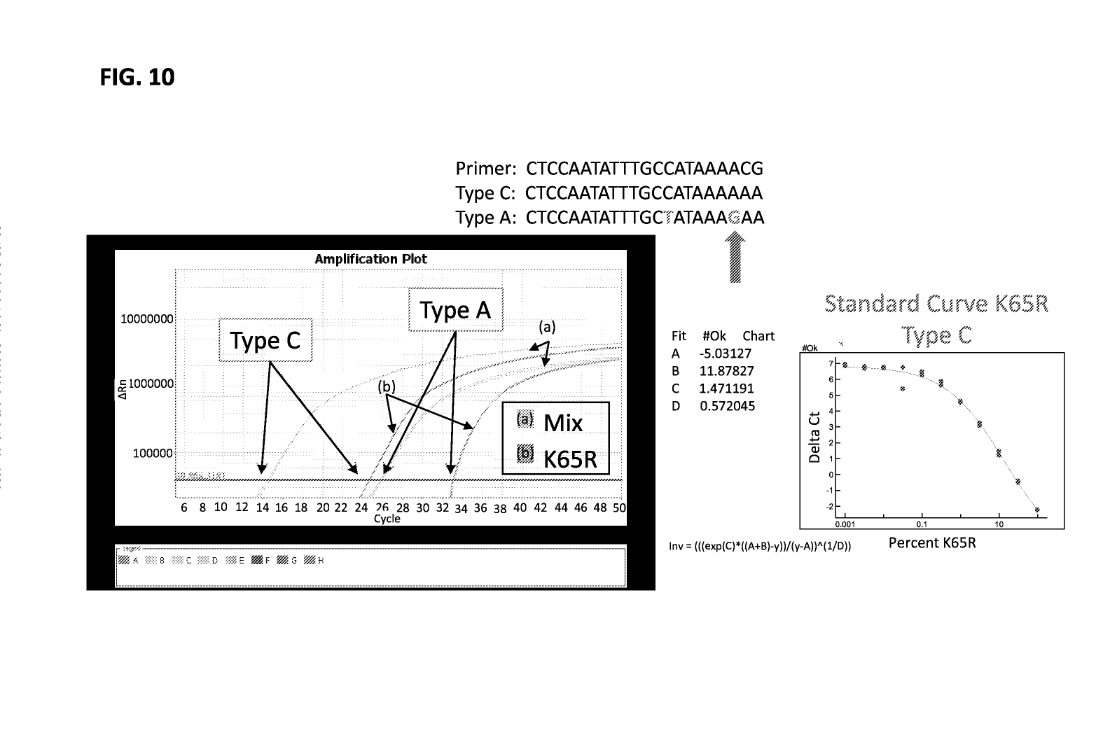

Due to the highly polymorphic nature of HIV, ASPCR is vulnerable to inaccurately estimating the concentration of an allele present and is prone to false positive as well as false negative results. Under current ASPCR methodologies, the allele specific PCR reactions against mutant or wild type virus template are normalized with a third qPCR reaction that estimates the total copy number (FIG. 3A). However, this total reaction can also be affected by HIV polymorphisms, displaying large differences on the amplification efficiencies observed with clinical samples. Alternatively, total copies can be calculated with a qPCR reaction in the same position as the allele specific reaction by moving the total primer upstream by one or two bases (FIG. 3A). Despite this approach, polymorphisms in proximity to the 3′ end of the allele specific primer (FIG. 3B), can still exert large effects on amplification efficiencies between allelic specific and total reaction, producing erroneous results. As illustrated in FIGS. 3C and 9, a new method for normalization was developed and tested, where the allele specific reaction was normalized against a PCR reaction that utilizes both the mutant and the wild type AS primers in one three primer or five primer PCR reaction (degenerative primer at the 3′ end position). The new method was tested using 10 clinical samples positive for subtype C HIV-1 virus and with available standard genotyping data (FIG. 6). Four of these samples were positive for the M184V mutation present at frequencies of 50-100% as determined by standard genotyping (ViroSeq; Celera). One of ten samples was positive for K65R mutation at a frequency of 20% and the remaining five samples were negative for the presence of the mutations at these two sites. ASPCR was performed using the PLUS primers and normalized with either the old or the new method. Normalization with the old method produced erroneous results for samples 9 and 10 characterized by the presence of M184V at a frequency greater than 100% (1,168 and 124%). Utilizing the new method of normalization, the frequency of M184V in samples 9 and 10 was calculated to be 51% and 44%, respectively. These values are in agreement with the estimated values based on their corresponding electropherograms, showing a clear improvement over the old method. Conversely, the presence of K65R in sample 1 was not detected by either the old or new method, despite its presence at a frequency of 20% by standard genotyping.

Further, as the new method is based on the Delta Ct it can compensate for differences between primers and template, for example in the case of different HIV-1 subtypes with varying polymorphisms. FIG. 9 illustrates an example of testing for K65R mutation in samples with subtype C HIV. Even though a sample with Type A virus has a G at −2 position adjacent to the penultimate mismatch, and amplification is delayed by 10 cycles, results are still translatable. In this example a Delta Ct of 8 is below the 0.1% cutoff of the assay or a Delta Ct of 7 (Range −3 to 7).

Reduction of Background and Limit of Detection with Proof Reading Polymerase.

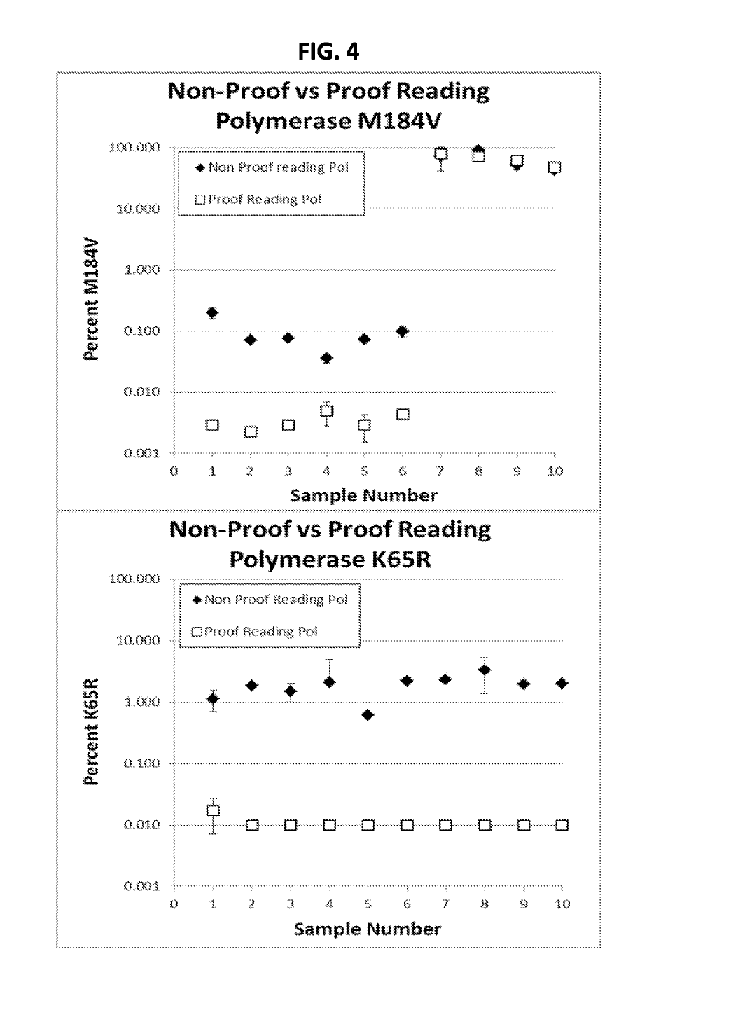

The absence of K65R in sample 1 was suggestive of a false positive, attributed to the utilization of a non-proof reading polymerase during first round amplification. In order to compensate for the possible introduction of errors during PCR, a proof reading polymerase during this initial amplification was used. The 10 clinical samples were retested by ASPCR for the presence of M184V and K65R using Phusion, a proof reading polymerase with a 3′ to 5′ exonuclease activity. A comparison between a non-proof reading (Taq) and a proof reading polymerase (Phusion) is illustrated in FIG. 4. The results obtained for the four positive M184V samples were concordant, between Taq and Phusion (Sample #7, 71.3% vs. 79.4%; Sample #8, 92.2% vs. 71.8%; Sample #9, 51.4% vs. 62.4%; and Sample 10, 43.6% vs. 48.0%), respectively. Furthermore, for samples negative for M184V as determined by standard genotyping, background levels decreased by 1 log (0.1% to values below 0.01%). More impressive were the results obtained for K65R, where a more than 2 log reduction was observed from 1% to below 0.01%, the limit of detection for this assay based on standards amplified from plasmids. As observed previously, Sample 1 was shown to be negative for K65R.

Validation Using Viral Mixtures.

The new ASPCR assay with the new primer design, new method of normalization, and the use of a proof-reading polymerase during the first round of amplification was validated using a blinded panel of 184V/wild-type viral mixtures spiked into negative human plasma at an average of 2.6×10⁵ copies/ml (FIG. 7). The values for 184V allele, as detected by ASPCR, were in accordance with expected values. A linear regression analysis of the input percent value of the mixtures versus the average calculated by ASPCR revealed a slope of 0.82 with an R² of 0.99. The CV for the negative sample was 36% and below 33% for all the positive samples. The limit of detection was down to 0.1%.

Detection of Linked Mutations Using ASPCR.

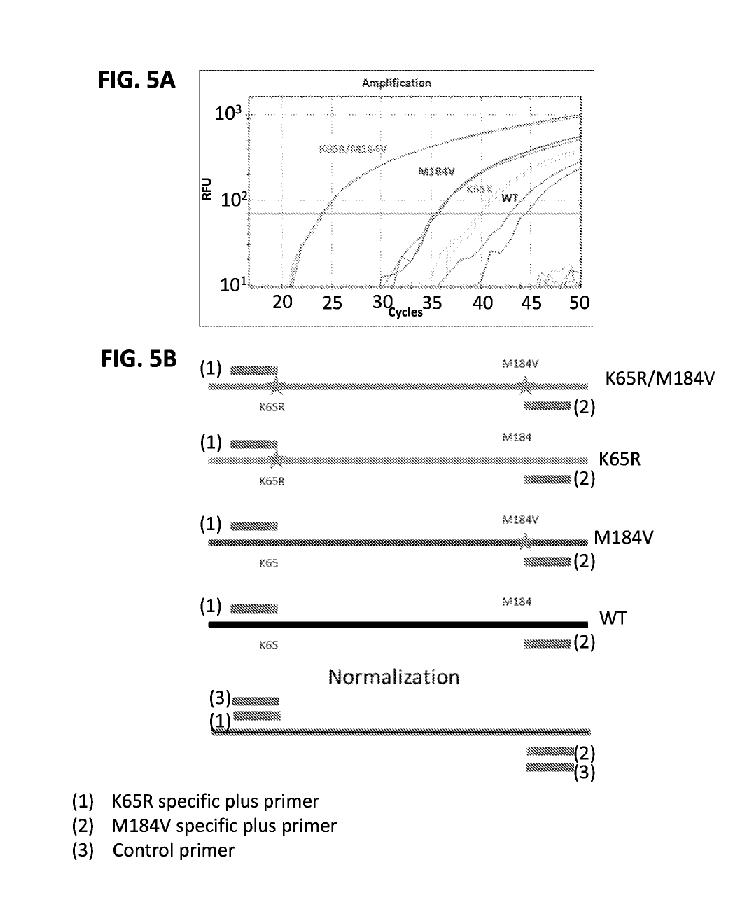

One limitation of current ASPCR technology is the detection of only one mutation at a time, limiting its application in the detection of linked drug-resistance mutations. However, the question of linkage can be addressed by the exploitation of our new methodology and the degenerative primer design (FIG. 3C), by utilizing allele specific primers for both the forward and reverse oligonucleotides used during 2^{nd} round PCR. This allows for the detection of two mutations at time and addresses the question of linkage. To test the concept of detecting linked mutations using ASPCR, standards were generated that harbored both the K65R and the M184V drug-resistance mutations. For the detection of these linked mutations the forward allele specific primer for K65R and the reverse allele specific primer for M184V were used, and tested in a qPCR reaction for their ability to discriminate between four standard templates; K65R/M184V (double mutant), K65R (single mutant), M184V (single mutant), or wild type (no mutant). As shown in FIG. 5A the double allele specific ASPCR had the ability to discriminate with a ΔCt of 11 cycles over the M184V template, a ΔCt of 16 cycles over the K65R template, and a ΔCt of 19 cycles over the wild type template. Using the new ASPCR method as depicted in FIG. 5B, and serial dilutions of standards of the double mutant in backgrounds of wild type, K65R, M184V, or mixtures of the three, showed a consistent detection limit below 0.1%, for all the different backgrounds used.

The assay was further validated using six clinical samples from patients that had failed first line therapy. Standard genotyping data were available for these six samples showing presence of K65R, M184V or both (FIG. 7). Quantification of linked K65R and M184V mutations was based on two standard curves, one with serial dilution of the double mutant in K65R background and one with serial dilutions of the double mutant in the M184V background. Linkage was detected in 5 out of the 6 samples, otherwise missed by standard genotyping (5 of 6 vs. 2 of 6). Calculated values of percent linked mutations were in concordance for both standard curves with either a K65R or M184V background.

### DISCUSSION

This example illustrates the development of an improved method for performing ASPCR for the detection of HIV-1 variants, including minor variants. The new method addresses issues associated with current ASPCR methods whose application in the clinical setting is limited. Improvements in current methodology includes the utilization of, a) a new primer design to increase sensitivity, b) a new method of normalization that minimizes variability associated with HIV polymorphism, c) employment of a proof reading polymerase to minimize false positives and a reduce background, and finally d) the ability to detect linkage between two drug resistance mutations, which is more relevant in a clinical setting than the detection of a single point mutation.

Currently, two primer designs have been recommended for their use in ASPCR to increase the discriminatory power of the assay between mutant and wild type templates. The first, described in the amplification refractory mutation system (ARMS), includes the introduction of a mismatch at the penultimate site (PEN), improving the assay for certain mismatch combinations that are poorly discriminated (Little et al., Curr. Protoc. Hum. Genet. 2001. Chapter 9: Unit 9.8. doi:10.1002/0471142905.hg0908s07, 2001). The second design incorporates a locked nucleic acid (LNA), a nucleic acid analog with a 2′-O, 4′-C methylene bridge at the 3′ end of the allele specific primer, which increases discrimination for all mismatch combinations (Latorra et al., Hum. Mutat. 22:79-85, 2003).

In an attempt to develop an ASPCR assay for the detection of the K65R mutation in subtype C HIV-1 virus, three different primer designs with identical sequences were compared which included LNA, PEN, and PLUS primers, the later a combination of the LNA and PEN primers, respectively (FIG. 1). The novel PLUS design improved the discriminatory power of the assay between the 65R and the 65K wild type templates characterized by a ΔCt increase of at least of 2 and 4 cycles when compared to PEN and LNA designs, respectively. Furthermore, despite these combined modifications, qPCR was only moderately compromised (PLUS Ct of 18 vs. Ct of 16 for PEN and LNA). This increased mismatch discrimination property associated with the PLUS primers was not unique for the K65R but also confirmed for the M814V and M184I ASPCR assay, suggesting that this is a generalized property. Surprisingly, for the M184V ASPCR, the ΔCt was more stable across a temperature gradient when comparing PLUS to PEN primers, suggesting that the increase in discrimination is not only due to an increase in Tm with the introduction of the LNA, but a restriction in the conformation of the 3′ end of primer may also play a role (Nielsen et al., J Biomol Struct Dyn 17:175-190, 1999).

Testing the prior ASPCR assay using with clinical samples revealed some erroneous results as shown in FIG. 6. One of the main hurdles for ASPCR is that the HIV genome is highly polymorphic and PCR efficiencies are altered from sample to sample depending on the number of mismatches that exists between the AS primer and the template. In order to compensate for this, the values from the original allele specific PCR reaction are normalized against a reaction that amplifies the total copies. Still, this total reaction is vulnerable to polymorphism, making very difficult to normalize the data accurately. Alternatively, utilization of primers that are at the same site as the allele specific primer missing the 3′ mismatch, even though is a good approximation, still can be affected by polymorphism. However, both approaches can succumb to the impact of polymorphisms as observed in FIG. 3B (i.e. −3 vs. −2 positions). To compensate for these discrepancies, investigators can introduce criteria to eliminate erroneous results (Boltz et al., PNAS. 22:9202-9207, 2011), or in cases that genotypic data is available, use patient-specific HIV consensus sequences for primer design (Rowley et al., J. Virol. Methods. 149:69-75, 2008; Boltz et al., J. Virol. Methods. 164:122-126, 2010).

In the new ASPCR method described in this example, the data are normalized against a total reaction that uses a degenerative primer at the 3′ end of the allele-specific primer and the same exact paired reverse or forward primer as in the ASPCR reaction. The advantage of the new method is several-fold. First, differences in the amplification efficiencies between the total and the allele-specific PCR can result only from the 3′ mismatch. Second, the new method is also more simplified, as for each sample two reactions are run in parallel to calculate the ΔCt of the total vs. the allele specific reaction. Third, the concentration of mutant is calculated based on the ΔCt of standards of known mixtures. Therefore, competition that exists in the total reaction is incorporated in the standards starting with a ΔCt of −1 for 100% mutant, with the allele specific reaction to be more efficient, to a ΔCt up to +15 for 100% wild type, depending the discriminatory power of the assay. As shown in FIG. 6, when we compared the new to the old method, observed vs. expected results were more concordant.

Validation of the K65R ASPCR assay using clinical samples showed that a sample that was positive for K65R by standard genotyping (AA/GG) was not detected and the negative result was confirmed by repeated testing either by additional ASPCR or standard genotyping. Utilization of a non-proof reading enzyme during first round amplification such as Taq, can result in such artifacts. Taq, with an error rate of 10⁻⁴ errors/base, lacks 3′-5′ exonuclease activity and is prone to of nucleotides, specifically A,T→G,C transitions, as is the case for K65R (AAG→AGG) (Tindall et al., Biochemistry. 27: 6008-6013, 1988). In addition, this effect can be compounded in samples with low viral loads where a misincorporation in the initial rounds of amplification can have a “jackpot effect” resulting in false positive results. Vargese et al. have reported similar results for spurious detection of K65R in PCR-dependent sequencing (Varghese et al., PLoS ONE 5(6):e10992. Doi:10.1371/journal.pone.0010992, 2010). These findings suggest that misincorporations during 1^{st} round PCR can prove to be a major issue, due to the increased sensitivity associated with ASPCR. Replacing Taq with Phusion, a proof-reading polymerase with a 3′ to 5′ exonuclease activity, was shown to have a large impact by a) eliminating false positives, b) reducing background, and c) increasing the sensitivity of the assay. This is evident in the case of K65R where the background of the assay with clinical samples was reduced by two logs from 1% down to 0.01%, values that were achieved before only with standards from plasmids, where proof reading is present.

Finally, using the new methodology for ASPCR the linkage of resistance mutations was addressed. The fact that the exact same amplicon was utilized as an internal control for normalization, provided the opportunity to include allelic specific primers for both the forward and the reverse primer, as shown in FIG. 5, and allowed for samples to be normalized with a PCR reaction using forward and reverse degenerative primers. The new assay was tested for the detection of K65R and M184V, mutations that are associated with resistance to TRUVADA (combination therapy of tenofovir (65R) and FTC (184V) (Masho et al., Ther. Clin. Risk Manag. 3:1097-1104, 2007). A template that has both resistance mutations was differentially amplified from the wild type template or the templates carrying either the individual K65R or M184V mutations separately. Discrimination for the M184V template was determined to be at a ΔCt of 11 cylces (the discriminatory power of the K65R allele specific primer), and for the K65R template a ΔCt of 16 (the discriminatory power of the M184V allele specific primer). Due to differences in discriminatory power between primers, the primer with the lower discriminatory power was set as the lower limit of sensitivity of the linked assay, which in this case was the K65R primer (0.1%). Furthermore, discrimination increases if the standards were run in a mixed background with any combination of K65R, M184V, or wild-type templates as well as all three templates together, while the ΔCt at the certain concentrations of standards remains the same. Applying the assay to test clinical samples from patients that had failed first line therapy revealed the presence of linked mutations in five out of six tested, while standard genotyping detected linkage in only in two of six. The absence of detection of linkage by standard genotyping in these three of six samples could be attributed to fitness of the virus. Previous reports have shown that the presence of both K65R and M184V mutations render the virus less fit which makes it difficult to maintain levels that can be detected with standard genotyping, in the absence of drug pressure (Deval et al., J. Biol. Chem. 279:509-516, 2004). Truvada is the most common prescribed combination drug for HIV that has also been recently approved for prophylaxis and detection of linked resistance to tenofovir and FTC at the sensitivity and cost that ASPCR offers could be very important.

In conclusion, this example describes an improved and simplified method for performing ASPCR with the potential to enter the clinic as a new diagnostic test for drug resistance either alone or in combination with a multiplexing technology. In addition, the methodology has the added advantage in that it can detect linked mutations, allowing for development of sensitive diagnostic resistance assays, tailored to specific therapies.

It will be apparent that the precise details of the methods or compositions described may be varied or modified without departing from the spirit of the described embodiments. We claim all such modifications and variations that fall within the scope and spirit of the claims below.

1. A method of detecting a mutant first allele of a target nucleic acid molecule in a biological sample, comprising:
(A) amplifying the target nucleic acid molecule from the biological sample by real-time polymerase chain reaction (qPCR) using a test primer pair in a test amplification, comprising
a first plus primer comprising a locked nucleic acid at the 3′ end that is complementary to the mutant first allele of the target nucleic acid molecule, a mismatch nucleotide at the −1 position from the 3′ end that is not complementary to the target nucleic acid molecule, and remaining nucleotides that are complementary to the target nucleic acid molecule; and
a reverse primer of the test primer pair;
(B) amplifying the target nucleic acid molecule from the biological sample by qPCR using a control set of primers in a control amplification, comprising
the first primer pair; and
a first control primer comprising a locked nucleic acid at the 3′ end that is complementary to a wildtype first allele corresponding to the mutant first allele, and remaining nucleotides that are the same as the first plus primer;
(C) determining a threshold cycle (Ct) value of the test amplification and a Ct value of the control amplification; and
(D) comparing a difference between the Ct values of the test and control amplifications with a standard control generated using a mixture of target nucleic acid molecules comprising a pre-selected proportion of the mutant and wildtype first alleles to detect the mutant first allele of the target nucleic acid molecule in the biological sample.

2. The method of claim 1, further comprising detecting a mutant second allele of the target nucleic acid molecule in the biological sample, wherein:
the reverse primer of the test primer pair is a second plus primer comprising a locked nucleic acid at the 3′ end that is complementary to the mutant second allele, a mismatch nucleotide at the −1 position from the 3′ end that is not complementary to the target nucleic acid molecule, and remaining nucleotides that are complementary to the target nucleic acid molecule;
the control set of primers further comprises a second control primer comprising a locked nucleic acid at the 3′ end that is complementary to a wildtype second allele corresponding to the mutant second allele, and remaining nucleotides that are the same as the second plus primer; and
the difference between the Ct values of the test and control amplifications are compared with a standard control generated using a mixture of target nucleic acid molecules comprising a pre-selected proportion of the mutant and wildtype first alleles and the mutant and wildtype second alleles to detect the mutant first and second alleles of the target nucleic acid molecule in the biological sample.

3. The method of claim 1, wherein the control set of primers further comprises:
a third control primer, comprising a locked nucleic acid at the 3′ end that is not complementary to the mutant or wildtype first allele, and remaining nucleotides that are the same as the first plus primer; and
a fourth control primer, comprising a locked nucleic acid at the 3′ end that is not complementary to the mutant or wildtype first allele and is not the same as the locked nucleic acid of the third control primer, and remaining nucleotides that are the same as the first plus primer.

4. The method of claim 2, wherein the control set of primers further comprises:
(a) a third control primer, comprising a locked nucleic acid at the 3′ end that is not complementary to the mutant or wildtype first allele, and remaining nucleotides that are the same as the first plus primer;
(b) a fourth control primer, comprising a locked nucleic acid at the 3′ end that is not complementary to the mutant or wildtype first allele and is not the same as the locked nucleic acid of the third control primer, and remaining nucleotides that are the same as the first plus primer;
(c) a fifth control primer, comprising a locked nucleic acid at the 3′ end that is not complementary to the mutant or wildtype second allele, and remaining nucleotides that are the same as the second plus primer;
(d) a sixth control primer, comprising a locked nucleic acid at the 3′ end that is not complementary to the mutant or wildtype second allele and is not the same as the locked nucleic acid of the fifth control primer, and remaining nucleotides that are the same as the second plus primer;
a combination of (a) and (b);
a combination of (c) and (d); or
a combination of (a), (b), (c), or (d).

5. The method of claim 1, wherein detecting the mutant first allele of the target nucleic acid molecule in the biological sample comprises detecting a proportion of the target nucleic acid molecules in the sample comprising the mutant first allele in the biological sample, and wherein no more than 20% of the target nucleic acid molecules in the biological sample comprise the mutant first allele.

6. The method of claim 5, wherein no more than 1% of the target nucleic acid molecules in the biological sample comprise the mutant first allele.

7. The method of claim 1, comprising a first round amplification of template DNA from the biological sample prior to the test or control amplifications, wherein the first round amplification comprises use of a proof-reading DNA polymerase.

8. The method of claim 1, wherein the mutant first allele, the mutant second allele, or both, is a drug resistant mutant allele of Human Immunodeficiency Virus (HIV)-1 or HIV-2.

9. The method of claim 8, wherein the mutant first allele, the mutant second allele, or both, is a drug resistant mutant allele of HIV-1 reverse transcriptase.

10. The method of claim 9, wherein the mutant first allele encodes one of the following mutations of HIV-1 reverse transcriptase: K65R, M184V, M184I, M41L, A62V, K65N, K65E, D67N, D67G, D67E, T69I, T69D, K70R, K70E, K70G, K70T, K70N, K70Q, L74V, L74I, V75I, V75M, V75T, F77L, L100I, K101P, K103N, K103S, V106M, Y115F, F116Y, Q151M, Y181C, Y181I, Y188L, G190S, G190A, L210W, T215Y, T215F, T215E, T215I, T215C, T215D, K219Q, K219E, K219N, K219R, P225H, or M230L.

11. The method of claim 9, wherein the first plus primer, the first control primer, and the reverse primer, comprise or consist of the nucleic acid sequences set forth as one of:
(a) SEQ ID NOs: 1, 2, and 3, respectively, for detecting a K65R allele;
(b) SEQ ID NOs: 29, 41, and 3, respectively, for detecting a K65R allele;
(c) SEQ ID NOs: 40, 42, and 3, respectively, for detecting a K65R allele;
(d) SEQ ID NOs: 1, 2, and 37, respectively, for detecting a K65R allele;
(e) SEQ ID NOs: 29, 41, and 37, respectively, for detecting a K65R allele;
(f) SEQ ID NOs: 40, 42, and 37, respectively, for detecting a K65R allele;
(g) SEQ ID NOs: 1, 2, and 38, respectively, for detecting a K65R allele;
(h) SEQ ID NOs: 29, 41, and 38, respectively, for detecting a K65R allele;
(i) SEQ ID NOs: 40, 42, and 38, respectively, for detecting a K65R allele;
(j) SEQ ID NOs: 4, 5 and 3, respectively, for detecting a K70E allele;
(k) SEQ ID NOs: 4, 5, and 37, respectively, for detecting a K70E allele;
(l) SEQ ID NOs: 4, 5, and 38, respectively, for detecting a K70E allele;
(m) SEQ ID NOs: 6, 7, and 8, respectively, for detecting a M184V allele;
(n) SEQ ID NOs: 9, 10, and 10, respectively, for detecting a M184I allele;
(o) SEQ ID NOs: 11, 12, and 14, respectively, for detecting a K103N allele;
(p) SEQ ID NOs: 15, 12, and 14, respectively, for detecting a K103N allele;
(q) SEQ ID NOs: 16, 17, and 17, respectively, for detecting a Y181C allele; or
(r) SEQ ID NOs: 19, 20, and 20, respectively, for detecting a G190A allele.

12. The method of claim 9, wherein the first and second alleles are selected from one of:
K65R and M184V, respectively;
K65R and M184I, respectively;
K65R and K103N, respectively;
K70E and M184V, respectively;
K70E and M184I, respectively;
K70E and K103N, respectively;
K103N and M184V, respectively; or
K103N and M184I, respectively.

13. The method of claim 2, wherein
(a) the first and second mutant alleles are K65R and M184V mutant alleles of HIV-1 reverse transcriptase, respectively, and wherein the first plus primer, the first control primer, the second plus primer, and the second control primer, comprise or consist of the nucleic acid sequences set forth as SEQ ID NOs: 1, 2, 6, and 7, respectively; or
(b) the first and second mutant alleles are K65R and M184I mutant alleles of HIV-1 reverse transcriptase, respectively, and wherein the first plus primer, the first control primer, the second plus primer, and the second control primer, comprise or consist of the nucleic acid sequences set forth as SEQ ID NOs: 1, 2, 9, and 10, respectively.

14. The method of claim 1, wherein the subject has an HIV-1 infection.

15. The method of claim 14, wherein the subject is being treated with a combination of tenofovir and emtricitabine.

16. The method of claim 8, further comprising identifying the subject as having a drug-resistant mutant allele of HIV-1 or HIV-2.

17. An isolated nucleic acid molecule, comprising a plus primer comprising or consisting of the nucleic acid sequence set forth as any one of SEQ ID NOs: 1-2, 4-5, 6-7, 9-12, 15-17, 19-20, or 39-42.

18. A composition, comprising:
(A) a primer pair comprising a forward and a reverse primer for amplifying a target nucleic acid molecule comprising a mutant first allele of a target nucleic acid; wherein
the forward primer is a first plus primer comprising a locked nucleic acid at the 3′ end that is complementary to the mutant first allele of the target nucleic acid molecule, a mismatch nucleotide at the −1 position from the 3′ end that is not complementary to the target nucleic acid molecule, and remaining nucleotides that are complementary to the target nucleic acid molecule; and
(B) a first control primer comprising a locked nucleic acid at the 3′ end that is complementary to a wildtype first allele corresponding to the mutant first allele, and remaining nucleotides that are the same as the first plus primer.

19. The composition of claim 18, wherein the first plus primer, the first control primer, and the reverse primer, comprise or consist of the nucleic acid sequences set forth as:
(a) SEQ ID NOs: 1, 2, and 3, respectively;
(b) SEQ ID NOs: 29, 41, and 3, respectively;
(c) SEQ ID NOs: 40, 42, and 3, respectively;
(d) SEQ ID NOs: 1, 2, and 37, respectively;
(e) SEQ ID NOs: 29, 41, and 37, respectively;
(f) SEQ ID NOs: 40, 42, and 37, respectively;
(g) SEQ ID NOs: 1, 2, and 38, respectively;
(h) SEQ ID NOs: 29, 41, and 38, respectively;
(i) SEQ ID NOs: 40, 42, and 38, respectively;
(j) SEQ ID NOs: 4, 5 and 3, respectively;
(k) SEQ ID NOs: 4, 5, and 37, respectively;
(l) SEQ ID NOs: 4, 5, and 38, respectively;
(m) SEQ ID NOs: 6, 7, and 8, respectively;
(n) SEQ ID NOs: 9, 10, and 10, respectively;
(o) SEQ ID NOs: 11, 12, and 14, respectively;
(p) SEQ ID NOs: 15, 12, and 14, respectively;
(q) SEQ ID NOs: 16, 17, and 17, respectively; or
(r) SEQ ID NOs: 19, 20, and 20, respectively.

20. The composition of claim 18, wherein the target nucleic acid molecule further comprises a second allele, and wherein
the reverse primer of the test primer pair is a second plus primer comprising a locked nucleic acid at the 3′ end that is complementary to the mutant second allele, a mismatch nucleotide at the −1 position from the 3′ end that is not complementary to the target nucleic acid molecule, and remaining nucleotides that are complementary to the target nucleic acid molecule; and
the composition further comprises a second control primer comprising a locked nucleic acid at the 3′ end that is complementary to a wildtype second allele corresponding to the mutant second allele, and remaining nucleotides that are the same as the second plus primer.

21. The composition of claim 20, wherein the first plus primer, the first control primer, the second plus primer, and the second control primer, comprise or consist of the nucleic acid sequences set forth as
SEQ ID NOs: 1, 2, 6, and 7, respectively; or
SEQ ID NOs: 1, 2, 9, and 10, respectively.

22. The composition of claim 18, further comprising:
a third control primer, comprising a locked nucleic acid at the 3′ end that is not complementary to the mutant or wildtype first allele, and remaining nucleotides that are the same as the first plus primer; and
the fourth control primer, comprising a locked nucleic acid at the 3′ end that is not complementary to the mutant or wildtype first allele and is not the same as the locked nucleic acid of the third control primer, and remaining nucleotides that are the same as the first plus primer.

23. The composition of claim 22, further comprising
(a) a third control primer, comprising a locked nucleic acid at the 3′ end that is not complementary to the mutant or wildtype first allele, and remaining nucleotides that are the same as the first plus primer;
(b) a fourth control primer, comprising a locked nucleic acid at the 3′ end that is not complementary to the mutant or wildtype first allele and is not the same as the locked nucleic acid of the third control primer, and remaining nucleotides that are the same as the first plus primer;
(c) a fifth control primer, comprising a locked nucleic acid at the 3′ end that is not complementary to the mutant or wildtype second allele, and remaining nucleotides that are the same as the second plus primer;
(d) a sixth control primer, comprising a locked nucleic acid at the 3′ end that is not complementary to the mutant or wildtype second allele and is not the same as the locked nucleic acid of the fifth control primer, and remaining nucleotides that are the same as the second plus primer;
a combination of (a) and (b);
a combination of (c) and (d); or
a combination of (a), (b), (c), or (d).


SEQUENCE LISTING (48 sequences)

SEQ ID NO: 1 - DNA, 22 bp, Artificial sequence
  oligonucleotide primer
ctccartatttgccataaaacg

SEQ ID NO: 2 - DNA, 22 bp, Artificial sequence
  oligonucleotide primer
ctccartatttgccataaaaca

SEQ ID NO: 3 - DNA, 21 bp, Artificial sequence
  oligonucleotide primer
tattcctaattgaacytccca

SEQ ID NO: 4 - DNA, 25 bp, Artificial sequence
  oligonucleotide primer
gccataaaaaagaaggaccagtacg

SEQ ID NO: 5 - DNA, 25 bp, Artificial sequence
  oligonucleotide primer
gccataaaaaagaaggaccagtaca

SEQ ID NO: 6 - DNA, 31 bp, Artificial sequence
  oligonucleotide primer
ctaagtcagatcctacatacaagtcatcccc

SEQ ID NO: 7 - DNA, 31 bp, Artificial sequence
  oligonucleotide primer
ctaagtcagatcctacatacaagtcatccct

SEQ ID NO: 8 - DNA, 29 bp, Artificial sequence
  oligonucleotide primer
tagtataaacaatgagacaccagggatta

SEQ ID NO: 9 - DNA, 38 bp, Artificial sequence
  oligonucleotide primer
ccctatttctaagtcagatcctacatacaaagtcatgt

SEQ ID NO: 10 - DNA, 38 bp, Artificial sequence
  oligonucleotide primer
ccctatttctaagtcagatcctacatacaaagtcatgc

SEQ ID NO: 11 - DNA, 27 bp, Artificial sequence
  oligonucleotide primer
cccacatctagtactgtcactgattga

SEQ ID NO: 12 - DNA, 27 bp, Artificial sequence
  oligonucleotide primer
cccacatctagtactgtcactgattgt

SEQ ID NO: 13 - DNA, 27 bp, Artificial sequence
  oligonucleotide primer
cccacatctagtactgtcactgattgc

SEQ ID NO: 14 - DNA, 29 bp, Artificial sequence
  oligonucleotide primer
aagtggagaaaattagtagatttcaggga

SEQ ID NO: 15 - DNA, 27 bp, Artificial sequence
  oligonucleotide primer
cccacatctagtactgtcactgattgg

SEQ ID NO: 16 - DNA, 27 bp, Artificial sequence
  oligonucleotide primer
ctacatacaagtcatccatatattgcc

SEQ ID NO: 17 - DNA, 27 bp, Artificial sequence
  oligonucleotide primer
ctacatacaagtcatccatatattgct

SEQ ID NO: 18 - DNA, 29 bp, Artificial sequence
  oligonucleotide primer
caccagggattagatatcaatataatgtg

SEQ ID NO: 19 - DNA, 28 bp, Artificial sequence
  oligonucleotide primer
ctatgttgccctatttctaagtcagagg

SEQ ID NO: 20 - DNA, 28 bp, Artificial sequence
  oligonucleotide primer
ctatgttgccctatttctaagtcagagc

SEQ ID NO: 21 - DNA, 23 bp, Artificial sequence
  oligonucleotide primer
aaacaatggccattgacagaaga

SEQ ID NO: 22 - DNA, 24 bp, Artificial sequence
  oligonucleotide primer
gttcataccccatccaaagaaatg

SEQ ID NO: 23 - DNA, 22 bp, Artificial sequence
  oligonucleotide primer
ctccartatttgccataaaacg

SEQ ID NO: 24 - DNA, 22 bp, Artificial sequence
  oligonucleotide primer
ctccartatttgccataaaaag

SEQ ID NO: 25 - DNA, 22 bp, Artificial sequence
  oligonucleotide primer
ctccartatttgccataaaaca

SEQ ID NO: 26 - DNA, 22 bp, Artificial sequence
  oligonucleotide primer
ctccartatttgccataaaaaa

SEQ ID NO: 27 - DNA, 31 bp, Artificial sequence; identical to SEQ ID NO: 6
  oligonucleotide primer

SEQ ID NO: 28 - DNA, 31 bp, Artificial sequence; identical to SEQ ID NO: 7
  oligonucleotide primer

SEQ ID NO: 29 - DNA, 37 bp, Artificial sequence
  oligonucleotide primer
ccctatttctaagtcagatcctacatacaagtcatgt

SEQ ID NO: 30 - DNA, 37 bp, Artificial sequence
  oligonucleotide primer
ccctatttctaagtcagatcctacatacaagtcatgc

SEQ ID NO: 31 - DNA, 34 bp, Artificial sequence
  oligonucleotide primer
ctatttctaagtcagatcctacatacaagtcatc

SEQ ID NO: 32 - DNA, 1680 bp, human immunodeficiency virus
cccattagccctattgagactgtaccagtaaaattaaagccaggaatggatggcccaaaagttaaacaatggccattgacagaagaaaaaataaaagcattagtagaaatttgtacagagatggaaaaggaagggaaaatttcaaaaattgggcctgaaaatccatacaatactccagtatttgccataaagaaaaaagacagtactaaatggagaaaattagtagatttcagagaacttaataagagaactcaagacttctgggaagttcaattaggaataccacatcccgcagggttaaaaaagaaaaaatcagtaacagtactggatgtgggtgatgcatatttttcagttcccttagatgaagacttcaggaagtatactgcatttaccatacctagtataaacaatgagacaccagggattagatatcagtacaatgtgcttccacagggatggaaaggatcaccagcaatattccaaagtagcatgacaaaaatcttagagccttttagaaaacaaaatccagacatagttatctatcaatacatggatgatttgtatgtaggatctgacttagaaatagggcagcatagaacaaaaatagaggagctgagacaacatctgttgaggtggggacttaccacaccagacaaaaaacatcagaaagaacctccattcctttggatgggttatgaactccatcctgataaatggacagtacagcctatagtgctgccagaaaaagacagctggactgtcaatgacatacagaagttagtggggaaattgaattgggcaagtcagatttacccagggattaaagtaaggcaattatgtaaactccttagaggaaccaaagcactaacagaagtaataccactaacagaagaagcagagctagaactggcagaaaacagagagattctaaaagaaccagtacatggagtgtattatgacccatcaaaagacttaatagcagaaatacagaagcaggggcaaggccaatggacatatcaaatttatcaagagccatttaaaaatctgaaaacaggaaaatatgcaagaatgaggggtgcccacactaatgatgtaaaacaattaacagaggcagtgcaaaaaataaccacagaaagcatagtaatatggggaaagactcctaaatttaaactgcccatacaaaaggaaacatgggaaacatggtggacagagtattggcaagccacctggattcctgagtgggagtttgttaatacccctcccttagtgaaattatggtaccagttagagaaagaacccatagtaggagcagaaaccttctatgtagatggggcagctaacagggagactaaattaggaaaagcaggatatgttactaatagaggaagacaaaaagttgtcaccctaactgacacaacaaatcagaagactgagttacaagcaatttatctagctttgcaggattcgggattagaagtaaacatagtaacagactcacaatatgcattaggaatcattcaagcacaaccagatcaaagtgaatcagagttagtcaatcaaataatagagcagttaataaaaaaggaaaaggtctatctggcatgggtaccagcacacaaaggaattggaggaaatgaacaagtagataaattagtcagtgctggaatcaggaaagtacta

SEQ ID NO: 33 - DNA, 22 bp, Artificial sequence
  oligonucleotide primer
ctccartatttgccataaaacc

SEQ ID NO: 34 - DNA, 22 bp, Artificial sequence
  oligonucleotide primer
ctccartatttgccataaaact

SEQ ID NO: 35 - DNA, 21 bp, Artificial sequence
  oligonucleotide primer
ctccartatttgccataaaaa

SEQ ID NO: 36 - protein, 560 aa, human immunodeficiency virus
PISPIETVPVKLKPGMDGPKVKQWPLTEEKIKALVEICTEMEKEGKISKIGPENPYNTPVFAIKKKDSTKWRKLVDFRELNKRTQDFWEVQLGIPHPAGLKKKKSVTVLDVGDAYFSVPLDEDFRKYTAFTIPSINNETPGIRYQYNVLPQGWKGSPAIFQSSMTKILEPFRKQNPDIVIYQYMDDLYVGSDLEIGQHRTKIEELRQHLLRWGLTTPDKKHQKEPPFLWMGYELHPDKWTVQPIVLPEKDSWTVNDIQKLVGKLNWASQIYPGIKVRQLCKLLRGTKALTEVIPLTEEAELELAENREILKEPVHGVYYDPSKDLIAEIQKQGQGQWTYQIYQEPFKNLKTGKYARMRGAHTNDVKQLTEAVQKITTESIVIWGKTPKFKLPIQKETWETWWTEYWQATWIPEWEFVNTPPLVKLWYQLEKEPIVGAETFYVDGAANRETKLGKAGYVTNRGRQKVVTLTDTTNQKTELQAIYLALQDSGLEVNIVTDSQYALGIIQAQPDQSESELVNQIIEQLIKKEKVYLAWVPAHKGIGGNEQVDKLVSAGIRKVL

SEQ ID NO: 37 - DNA, 21 bp, Artificial sequence
  oligonucleotide primer
tattcctaattgaacctccca

SEQ ID NO: 38 - DNA, 21 bp, Artificial sequence
  Oligonucleotide primer
tattcctaattgaacttccca

SEQ ID NO: 39 - DNA, 22 bp, Artificial sequence
  Oligonucleotide primer
ctccaatatttgccataaaacg

SEQ ID NO: 40 - DNA, 22 bp, Artificial sequence
  Oligonucleotide primer
ctccagtatttgccataaaacg

SEQ ID NO: 41 - DNA, 22 bp, Artificial sequence
  oligonucleotide primer
ctccaatatttgccataaaaca

SEQ ID NO: 42 - DNA, 22 bp, Artificial sequence
  oligonucleotide primer
ctccagtatttgccataaaaca

SEQ ID NO: 43 - DNA, 22 bp, Artificial sequence
  oligonucleotide primer
ctccaatatttgccataaaacc

SEQ ID NO: 44 - DNA, 22 bp, artificial sequence
  oligonucleotide primer
ctccaatatttgccataaaact

SEQ ID NO: 45 - DNA, 22 bp, artificial sequence
  oligonucleotide primer
ctccagtatttgccataaaacc

SEQ ID NO: 46 - DNA, 22 bp, artificial sequence
  oligonucleotide primer
ctccagtatttgccataaaact

SEQ ID NO: 47 - DNA, 22 bp, Artificial sequence
  oligonucleotide primer
ctccaatatttgccataaaaaa

SEQ ID NO: 48 - DNA, 22 bp, Artificial Sequence
  oligonucleotide primer
ctccaatatttgctataaagaa